SEQUENCE LISTING (48 sequences)

SEQ ID NO: 1 - DNA, 627 bp, Artificial Sequence
  KGF-2 cDNA
atgtggaaatggatactgacacattgtgcctcagcctttccccacctgcccggctgctgctgctgctgctttttgttgctgttcttggtgtcttccgtccctgtcacctgccaagcccttggtcaggacatggtgtcaccagaggccaccaactcttcttcctcctccttctcctctccttccagcgcgggaaggcatgtgcggagctacaatcaccttcaaggagatgtccgctggagaaagctattctctttcaccaagtactttctcaagattgagaagaacgggaaggtcagcgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 2 - protein, 208 aa, Artificial Sequence
  KGF-2 cDNA
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 3 - DNA, 444 bp, Artificial Sequence
  KGF-2 28 cDNA
atggctggtcgtcacgttcgttcttacaaccacctgcagggtgacgttcgttggcgtaaactgttctctttcaccaaatacttcctgaaaatcgaaaaaaacggtaaagtttctgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 4 - protein, 147 aa, Artificial Sequence
  KGF-2 28 cDNA
MAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 5 - DNA, 582 bp, Artificial Sequence
  FGF-12 cDNA
atgcagggggagaatcacccgtctcctaattttaaccagtacgtgcgtgaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacg

SEQ ID NO: 6 - protein, 181 aa, Artificial Sequence
  FGF-12
MESKEPQLKGIVTRLFSQQGYFLQMHPDGTIDGTKDENSDYTLFNLIPVGLRVVAIQGVKASLYVAMNGEGYLYSSDVFTPECKFKESVFENYYVIYSSTLYRQQESGRAWFLGLNKEGQIMKGNRVKKTKPSSHFVPKPIEVCMYREPSLHEIGEKQGRSRKSSGTPTMNGGKVVNQDST

SEQ ID NO: 7 - DNA, 627 bp, Artificial Sequence
  E. coli optimized full length KGF-2 cDNA
atgtggaaatggatactgacccactgcgcttctgctttcccgcacctgccgggttgctgctgctgctgcttcctgctgctgttcctggtttcttctgttccggttacctgccaggctctgggtcaggacatggtttctccggaagctaccaactcttcctcttcctctttctcttccccgacttccgctggtcgtcacgttcgttcttacaaccacctgcagggtgacgttcgttggcgtaaactgttctctttcaccaaatacttcctgaaaatcgaaaaaaacggtaaagtttctgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 8 - protein, 208 aa, Artificial Sequence
  E. coli optimized full length KGF-2 cDNA
MWKWILTHCASAFPHLPGCCCCCFLLLFLVSSVPVTCQALGQDMVSPEATNSSSSSFSSPTSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 9 - DNA, 525 bp, Artificial Sequence
  E. coli optimized mature KGF-2 cDNA
atgacctgccaggctctgggtcaggacatggtttctccggaagctaccaactcttcctcttcctctttctcttccccgtcttccgctggtcgtcacgttcgttcttacaaccacctgcagggtgacgttcgttggcgtaaactgttctctttcaccaaatacttcctgaaaatcgaaaaaaacggtaaagtttctgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 10 - protein, 174 aa, Artificial Sequence
  E. coli optimized mature KGF-2 cDNA
MTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 11 - DNA, 525 bp, Artificial Sequence
  alternate E. coli optimized mature KGF-2 cDNA
atgacttgccaggcactgggtcaagacatggtttccccggaagctaccaacagctccagctctagcttcagcagcccatctagcgcaggtcgtcacgttcgctcttacaaccacttacagggtgatgttcgttggcgcaaactgttcagctttaccaagtacttcctgaaaatcgaaaaaaacggtaaagtttctgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 12 - protein, 174 aa, Artificial Sequence; identical to SEQ ID NO: 10
  alternate E. coli optimized mature KGF-2 cDNA

SEQ ID NO: 13 - DNA, 36 bp, Artificial Sequence
  Oligonucleotide primer
ccccacatgtggaaatggatactgacacattgtgcc

SEQ ID NO: 14 - DNA, 35 bp, Artificial Sequence
  Oligonucleotide primer
cccaagcttccacaaacgttgccttcctctatgag

SEQ ID NO: 15 - DNA, 84 bp, Artificial Sequence
  Oligonucleotide primer
atgtggaaatggatactgacccactgcgcttctgctttcccgcacctgccgggttgctgctgctgctgcttcctgctgctgttc

SEQ ID NO: 16 - DNA, 82 bp, Artificial Sequence
  Oligonucleotide primer
ccggagaaaccatgtcctgacccagagcctggcaggtaaccggaacagaagaaaccaggaacagcagcaggaagcagcagca

SEQ ID NO: 17 - DNA, 80 bp, Artificial Sequence
  Oligonucleotide primer
gggtcaggacatggtttctccggaagctaccaactcttcttcttcttctttctcttctccgtcttctgctggtcgtcacg

SEQ ID NO: 18 - DNA, 81 bp, Artificial Sequence
  Oligonucleotide primer
ggtgaaagagaacagtttacgccaacgaacgtcaccctgcaggtggttgtaagaacgaacgtgacgaccagcagaagacgg

SEQ ID NO: 19 - DNA, 75 bp, Artificial Sequence
  Oligonucleotide primer
cgttggcgtaaactgttctctttcaccaaatacttcctgaaaatcgaaaaaaacggtaaagtttctgggaccaaa

SEQ ID NO: 20 - DNA, 39 bp, Artificial Sequence
  Oligonucleotide primer
tttggtcccagaaactttaccgtttttttcgattttcag

SEQ ID NO: 21 - DNA, 36 bp, Artificial Sequence
  Oligonucleotide primer
aaaggatccatgtggaaatggatactgacccactgc

SEQ ID NO: 22 - DNA, 38 bp, Artificial Sequence
  Oligonucleotide primer
tttcatgacttgtcaagctctgggtcaagatatggttc

SEQ ID NO: 23 - DNA, 28 bp, Artificial Sequence
  Oligonucleotide primer
gcccaagcttccacaaacgttgccttcc

SEQ ID NO: 24 - DNA, 45 bp, Artificial Sequence
  Oligonucleotide primer
tcagtgaattcattaaagaggagaaattaatcatgacttgccagg

SEQ ID NO: 25 - DNA, 48 bp, Artificial Sequence
  Oligonucleotide primer
tcatgacttgccaggcactgggtcaagacatggtttccccggaagcta

SEQ ID NO: 26 - DNA, 48 bp, Artificial Sequence
  Oligonucleotide primer
gcttcagcagcccatctagcgcaggtcgtcacgttcgctcttacaacc

SEQ ID NO: 27 - DNA, 48 bp, Artificial Sequence
  Oligonucleotide primer
gttcgttggcgcaaactgttcagctttaccaagtacttcctgaaaatc

SEQ ID NO: 28 - DNA, 28 bp, Artificial Sequence
  Oligonucleotide primer
tcgaaaaaaacggtaaagtttctgggac

SEQ ID NO: 29 - DNA, 48 bp, Artificial Sequence
  Oligonucleotide primer
gatgggctgctgaagctagagctggagctgttggtagcttccggggaa

SEQ ID NO: 30 - DNA, 45 bp, Artificial Sequence
  Oligonucleotide primer
aacagtttgcgccaacgaacatcaccctgtaagtggttgtaagag

SEQ ID NO: 31 - DNA, 47 bp, Artificial Sequence
  Oligonucleotide primer
ttcttggtcccagaaactttaccgtttttttcgattttcaggaagta

SEQ ID NO: 32 - DNA, 24 bp, Artificial Sequence
  Oligonucleotide primer
ttcttggtcccagaaactttaccg

SEQ ID NO: 33 - DNA, 45 bp, Artificial Sequence
  Oligonucleotide primer
agatcaggcttctattattatgagtgtaccaccattggaagaaag

SEQ ID NO: 34 - DNA, 35 bp, Artificial Sequence
  Oligonucleotide primer
ggaccctcatgacctgccaggctctgggtcaggac

SEQ ID NO: 35 - DNA, 28 bp, Artificial Sequence
  Oligonucleotide primer
ggacagccatggctggtcgtcacgttcg

SEQ ID NO: 36 - DNA, 37 bp, Artificial Sequence
  Oligonucleotide primer
ctgcccaagcttattatgagtgtaccaccattggaag

SEQ ID NO: 37 - DNA, 36 bp, Artificial Sequence
  Oligonucleotide primer
ctgcccaagcttattacttcagcttacagtcattgt

SEQ ID NO: 38 - DNA, 29 bp, Artificial Sequence
  Oligonucleotide primer
ggaccctcatgacctctcaggctctgggt

SEQ ID NO: 39 - DNA, 21 bp, Artificial Sequence
  Oligonucleotide primer
aaggagaactctccgtacagc

SEQ ID NO: 40 - DNA, 21 bp, Artificial Sequence
  Oligonucleotide primer
gctgtacggtctgttctcctt

SEQ ID NO: 41 - DNA, 35 bp, Artificial Sequence; identical to SEQ ID NO: 34
  Oligonucleotide primer

SEQ ID NO: 42 - DNA, 37 bp, Artificial Sequence; identical to SEQ ID NO: 36
  Oligonucleotide primer

SEQ ID NO: 43 - DNA, 456 bp, Artificial Sequence
  pHE4KGF-2.A63-S208
catatggctggtcgtcacgttcgttcttacaaccacctgcagggtgacgttcgttggcgtaaactgttctctttcaccaaatacttcctgaaaatcgaaaaaaacggtaaagtttctgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcataataaggtacc

SEQ ID NO: 44 - DNA, 48 bp, Artificial Sequence
  Oligonucleotide primer
gactacatatggctggtcgtcacgttcgttcttacaaccacctgcagg

SEQ ID NO: 45 - DNA, 47 bp, Artificial Sequence
  Oligonucleotide primer
ctagtctctagattattatgagtgtacaaccatcggcaggaagtgag

SEQ ID NO: 46 - DNA, 447 bp, Artificial Sequence
  pHE4KGF-2.A63-S208
atggctggtcgtcacgttcgttcttacaaccacctgcagggtgacgttcgttggcgtaaactgttctctttcaccaaatacttcctgaaaatcgaaaagaacggtaaagtttctggtaccaagaaagaaaactgcccgtactctatcctggaaatcacctccgttgaaatcggtgttgtagccgttaaagccatcaactccaactattacctggccatgaacaaaaagggtaaactgtacggctctaaagaattcaacaacgactgcaaactgaaagaacgtatcgaagagaacggttacaacacctacgcatccttcaactggcagcacaacggtcgtcagatgtacgttgcactgaacggtaaaggcgctccgcgtcgcggtcagaaaacccgtcgcaaaaacacctctgctcacttcctgccgatggttgtacactcataataa

SEQ ID NO: 47 - DNA, 591 bp, Artificial Sequence
  FGF-13 cDNA
atgcagggggagaatcacccgtctcctaattttaaccagtacgtgcgtgaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgarggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacgtaaggtacc

SEQ ID NO: 48 - protein, 194 aa, Artificial Sequence
  FGF-13 cDNA
MQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

(19) United States
(12) Patent Application Publication
(10) Pub. No.: US 2003/0119108 A1
(43) Pub. Date: Jun. 26, 2003

(54) Method of making FGF-12 and KGF-2

(75) Inventor: Michael W. Laird, Germantown, MD (US)

Correspondence Address: STERNE, KESSLER, GOLDSTEIN & FOX P.L.L.C., 1100 NEW YORK AVENUE, N.W., SUITE 600, WASHINGTON, DC, 20005-3934, US

(73) Assignee: Human Genome Sciences, Inc.

(21) Appl. No.: 10/194,443

(22) Filed: Jul. 12, 2002

Related U.S. Application Data
(60) Provisional application No. 60/304,642, filed on Jul. 12, 2001.

(51) Int. Cl. C12P 21/02; C12N 1/21; C12N 9/00; C07H 21/04; C07K 14/475

(52) U.S. Cl. 435/69.1; 435/183; 435/320.1; 435/325; 435/252.33; 536/23.2; 530/399

(57) ABSTRACT

The present invention is directed to a method of expressing KGF-2 polypeptides in an ompT-deficient prokaryotic host cell. Also provided are ompT-deficient prokaryotic host cells comprising polynucleotides encoding KGF-2. The present invention is further directed to a method of expressing FGF-12 in an ompT-deficient prokaryotic host cell. Also provided are ompT-deficient prokaryotic hot cells comprising polynucleotides encoding FGF-12.

### CROSS-REFERENCE TO RELATED APPLICATIONS

This application claims priority benefit of U.S. Provisional Application No. 60/304,642, filed Jul. 12, 2001, the entire contents of which are incorporated by reference herein.

### FIELD OF THE INVENTION

This invention relates to genetically engineered host cells and protein expression. More particularly, the invention relates to ompT-deficient prokaryotic hosts genetically engineered with a polynucleotide encoding a Keratinocyte Growth Factor-2 (KGF-2) polypeptide, and methods of producing the KGF-2 using such host cells. This invention also related to ompT-deficient prokaryotic hosts genetically engineered with a polynucleotide encoding Fibroblast Growth Factor-12 (FGF-12), and methods of producing FGF-12 using such host cells.

### BACKGROUND ART

The fibroblast growth factor family has emerged as a large family of growth factors involved in soft-tissue growth and regeneration. It presently includes several members that share a varying degree of homology at the protein level, and that, with one exception, appear to have a similar broad mitogenic spectrum, i.e., they promote the proliferation of a variety of cells of mesodermal and neuroectodermal origin and/or promote angiogenesis.

KGF was originally identified as a member of the FGF family by sequence homology or factor purification and cloning. Keratinocyte growth factor (KGF) was isolated as a mitogen from a cultured murine keratinocyte line (Rubin, J. S. et al., Proc. Natl. Acad. Sci. USA 86:802-806 (1989)). Unlike the other members of the FGF family, it has little activity on mesenchyme-derived cells but stimulates the growth of epithelial cells. Keratinocyte growth factor is produced by fibroblasts derived from skin and fetal lung (Rubin et al. (1989)). The Keratinocyte growth factor mRNA was found to be expressed in adult kidney, colon and ilium, but not in brain or lung (Finch, P. W. et al., Science 245:752-755 (1989)). KGF displays the conserved regions within the FGF protein family. KGF binds to the FGF-2 receptor with high affinity.

Keratinocyte Growth Factor-2 (KGF-2), also known as Fibroblast Growth Factor-10 (FGF-10), was identified as a member of the FGF family (Igarashi et al., J. Biol. Chem. 273(1):13230-13235 (1998)). KGF-2 has been shown to be a growth factor for keratinocytes and to promote wound healing (Soler et al., Wound Repair Regen. 7(3):172-178 (1999); and Marchese et al., J. Invest. Dermatol. 116(4):623-628 (2001)).

Impaired wound healing is a significant source of morbidity and may result in such complications as dehiscence, anastomotic breakdown and, non-healing wounds. In the normal individual, wound healing is achieved uncomplicated. In contrast, impaired healing is associated with several conditions such as diabetes, infection, immunosuppression, obesity and malnutrition (Cruse, P. J. and Foord, R., Arch. Surg. 107:206 (1973); Schrock, T. R. et al., Ann. Surg. 177:513 (1973); Poole, G. U., Jr., Surgery 97:631 (1985); Irvin, G. L. et al., Am. Surg. 51:418 (1985)).

### BRIEF SUMMARY OF THE INVENTION

The present invention is directed to an ompT-deficient prokaryotic host cell which is genetically engineered with a polynucleotide encoding a KGF-2 polypeptide.

The present invention is also directed to a method of producing a KGF-2 polypeptide comprising culturing a host cell of the present invention under conditions sufficient to induce expression of said polypeptide, and recovering said polypeptide.

The present invention is also directed to an ompT-deficient prokaryotic host cell which is genetically engineered with a polynucleotide encoding FGF-12.

The present invention is also directed to a method of producing a FGF-12 polypeptide comprising culturing a host cell of the present invention under conditions sufficient to induce expression of said polypeptide, and recovering said polypeptide.

### BRIEF DESCRIPTION OF THE DRAWINGS/FIGURES

FIG. 1 shows the DNA and the encoded protein sequence (SEQ ID NOS:1 and 2) for KGF-2.

FIG. 2 shows the DNA and the encoded protein sequence (SEQ ID NOS:3 and 4) for KGF-2Δ28.

FIG. 3 shows the DNA and the encoded protein sequence (SEQ ID NO:5 and 6) for FGF-12.

FIG. 4 shows the DNA and the encoded protein sequence (SEQ ID NO:7 and 8) of the E. coli optimized full length KGF-2.

FIG. 5 shows the DNA and the encoded protein sequence (SEQ ID NO:9 and 10) of the E. Coli optimized mature KGF-2.

FIG. 6 shows the DNA and the encoded protein sequence (SEQ ID NO:11 and 12) of the alternate E. coli optimized mature KGF-2.

FIG. 7 shows the DNA and the encoded protein sequence (SEQ ID NO:47 and 48) for FGF-13.

### DETAILED DESCRIPTION OF THE INVENTION

OmpT is a 36 kD outer-membrane protease found in bacterial cells. The present inventors have found that OmpT is responsible for degradation of KGF-2 polypeptide when KGF-2 is expressed in bacterial cells. OmpT cleaves KGF-2 between amino acid residues Arg (68) and Ser (69).

The present inventors have also found that OmpT is responsible for the degradation of FGF-12 when FGF-12 is expressed in bacterial cells. FGF-12 has an ompT cleavage site 19 amino acids in from the C-terminal end of the protein.

Thus, the present invention provides an ompT-deficient prokaryotic host cell comprising a polynucleotide encoding a KGF-2 polypeptide. The present invention also provides an ompT-deficient prokaryotic host cell comprising a polynucleotide encoding FGF-12 or a fragment or variant thereof. The present invention also provides an ompT-deficient prokaryotic host cell comprising a polynucleotide encoding FGF-13 or a fragment or variant thereof.

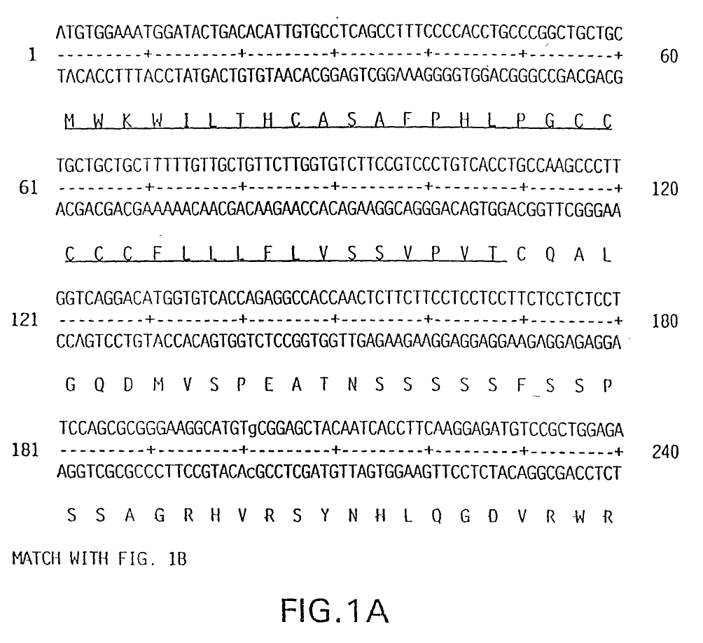

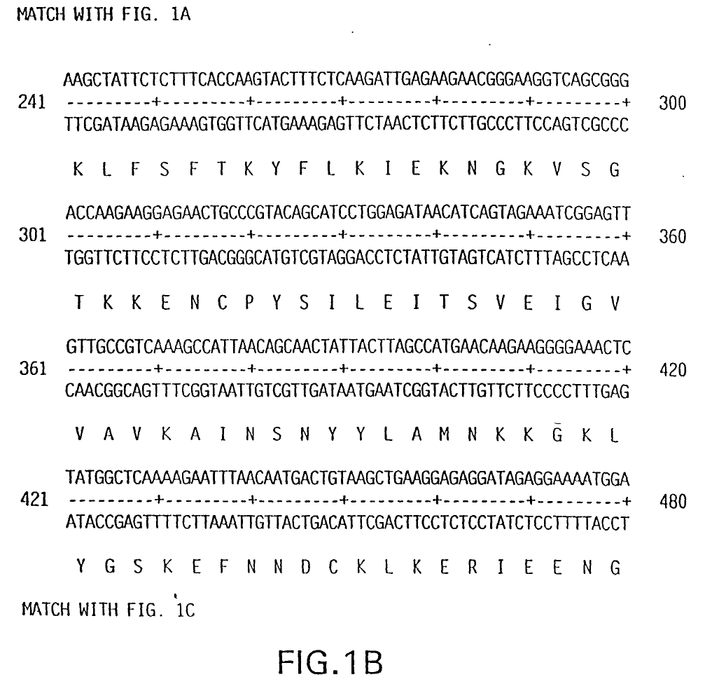

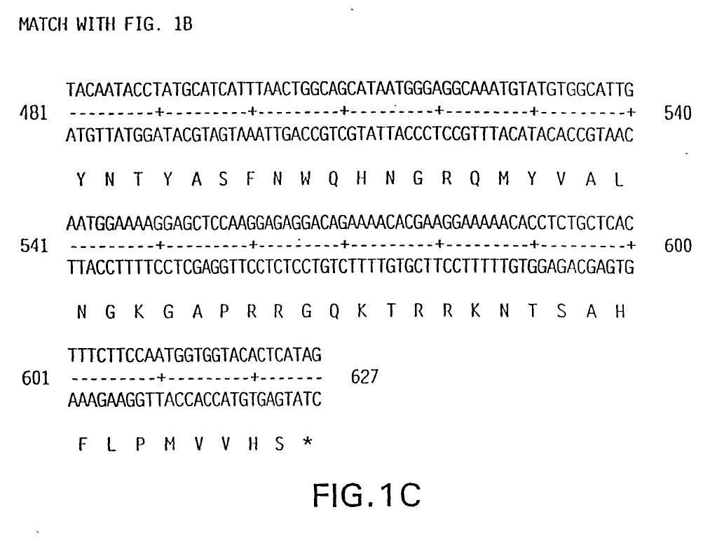

In accordance with an aspect of the present invention, there is provided an isolated nucleic acid (polynucleotide) which encodes for the polypeptide having the deduced amino acid sequence of FIG. 1 (SEQ ID NO:2) or for the polypeptide encoded by the cDNA of the clone deposited as ATCC Deposit No. 75977 on Dec. 16, 1994 at the American Type Culture Collection Patent Depository, 10801 University Boulevard, Manassas, Va. 20110-2209 or the polypeptide encoded by the cDNA of the clone deposited as ATCC Deposit No. 75901 on Sep. 29, 1994 at the American Type Culture Collection Patent Depository, 10801 University Boulevard, Manassas, Va. 20110-2209. The nucleotide sequence determined by sequencing the deposited KGF-2 clone, which is shown in FIG. 1 (SEQ ID NO:1), contains an open reading frame encoding a polypeptide of 208 amino acid residues, including an initiation codon at positions 1-3, with a predicted leader sequence of about 35 or 36 amino acid residues, and a deduced molecular weight of about 23.4 kDa. The amino acid sequence of the mature KGF-2 is shown in FIG. 1, amino acid residues about 36 or 37 to 208 (SEQ ID NO:2).

A cDNA encoding the KGF-2Δ28 polypeptide, optimized for expression in E. coli, was deposited as ATCC Deposit No. PTA-2184 on Jul. 3, 2000 at the American Type Culture Collection Patent Depository, 10801 University Boulevard, Manassas, Va. 20110-2209. A cDNA encoding the KGF-2Δ28 polypeptide plus an N-terminal methionine, optimized for expression in E. coli, was deposited as ATCC Deposit No. PTA-2183 on Jul. 3, 2000 at the American Type Culture Collection Patent Depository, 10801 University Boulevard, Manassas, Va. 20110-2209.

Thus, the present invention is directed to a method of producing KGF-2 or FGF-12 polypeptides. In particular, the invention is directed to a method for producing a KGF-2 polypeptide comprising culturing an ompT deficient prokaryotic host cell genetically engineered with an isolated polynucleotide encoding KGF-2 under conditions sufficient to express the polypeptide, and recovering the polypeptide. The invention is also directed to a method for producing a FGF-12 polypeptide comprising culturing an ompT deficient prokaryotic host cell genetically engineered with an isolated polynucleotide encoding FGF-12 under conditions sufficient to express the polypeptide, and recovering the polypeptide. Preferred host cells are bacteria, including gram-negative bacteria. Particularly preferred are Haemophilis, Pseudomonas, Klebsiella, E. coli and Salmonella.

By “ompT-deficient” it is meant that the function of the OmpT protein is impaired in some manner. The ompT gene may be completely or partially deleted, or one or more sequences controlling ompT expression maybe completely or partially deleted. Additionally, the translation of OmpT protein may be impaired. In addition, the protein may be expressed, but in mutated form such that the protein does not function to cleave the KGF-2 or FGF-12 polypeptides. These lesions include mutations of single or multiple nucleotide residues, deletions, and/or insertions.

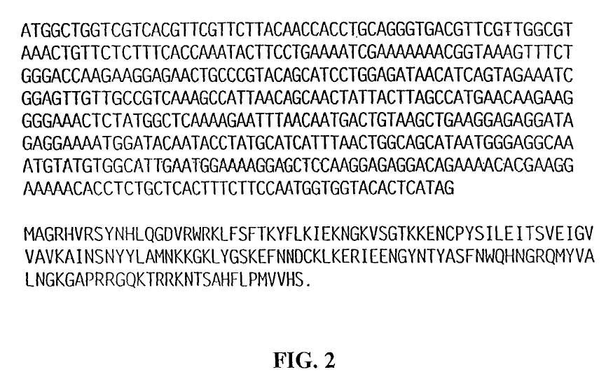

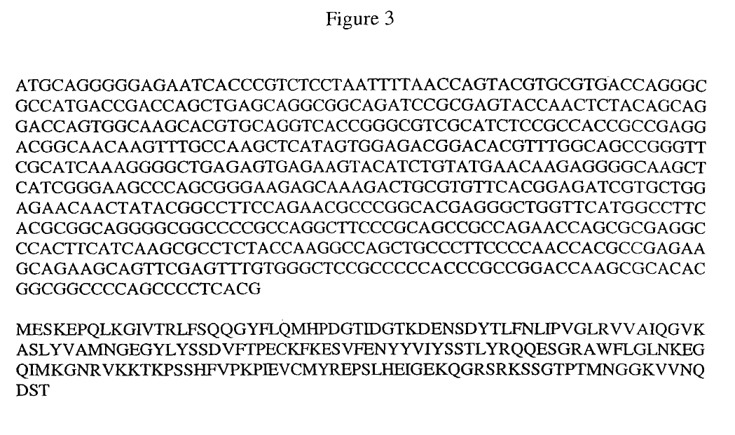

A nucleotide and amino acid sequence of KGF-2 is provided in FIG. 1 (SEQ ID NOs:1 and 2). A truncated version of KGF-2, KGF-2Δ28, is provided in FIG. 2 (SEQ ID NOs:3 and 4). A nucleotide and amino acid sequence of FGF-12 is provided in FIG. 3 (SEQ ID NOs:5 and 6).

Isolated nucleic acid molecules useful in the method and hosts of the present invention include DNA molecules comprising an open reading frame (ORF) with an initiation codon at positions 1-3 of the nucleotide sequence shown in FIG. 1 (SEQ ID NO:1); DNA molecules comprising the coding sequence for the mature KGF-2 protein shown in FIG. 1 (last 172 or 173 amino acids) (SEQ ID NO:2); and DNA molecules which comprise a sequence substantially different from those described above but which, due to the degeneracy of the genetic code, still encode the KGF-2 protein. Of course, the genetic code is well known in the art. Thus, it would be routine for one skilled in the art to generate the degenerate variants described above.

Unless otherwise indicated, each “nucleotide sequence” set forth herein is presented as a sequence of deoxyribonucleotides (abbreviated A, G, C and T). However, by “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U). For instance, reference to an RNA molecule having the sequence of SEQ ID NO:1 set forth using deoxyribonucleotide abbreviations is intended to indicate an RNA molecule having a sequence in which each deoxyribonucleotide A, G or C of SEQ ID NO:1 has been replaced by the corresponding ribonucleotide A, G or C, and each deoxyribonucleotide T has been replaced by a ribonucleotide U.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) with an initiation codon at positions 1-3 of the nucleotide sequence shown in FIG. 1 (SEQ ID NO:1); DNA molecules comprising the coding sequence for the mature KGF-2 protein shown in FIG. 1 (last 172 or 173 amino acids) (SEQ ID NO:2); DNA molecules comprising the nucleotide sequence shown in FIG. 3 (SEQ ID NO:5); and DNA molecules which comprise a sequence substantially different from those described above but which, due to the degeneracy of the genetic code, still encode the KGF-2 or FGF-12 protein. Of course, the genetic code is well known in the art. Thus, it would be routine for one skilled in the art to generate the degenerate variants described above.

Polynucleotides useful in the present invention may be in the form of RNA or in the form of DNA, which DNA includes cDNA, genomic DNA, and synthetic DNA. The DNA may be doublestranded or single-stranded, and if single stranded may be the coding strand or non-coding (anti-sense) strand. The coding sequence which encodes the KGF-2 polypeptide may be identical to the coding sequence shown in FIG. 1 (SEQ ID NO:1) or that of the deposited clone or may be a different coding sequence which coding sequence, as a result of the redundancy or degeneracy of the genetic code, encodes the same polypeptide as the DNA of FIG. 1 (SEQ ID NO:1) or the deposited cDNA. The coding sequence which encodes the FGF-12 polypeptide may be identical to the coding sequence shown in FIG. 3 (SEQ ID NO:5) or may be a different coding sequence which coding sequence, as a result of the redundancy or degeneracy of the genetic code, encodes the same polypeptide as the DNA of FIG. 3 (SEQ ID NO:5).

The polynucleotide which encodes for the polypeptide of FIG. 1 (SEQ ID NO:2) or for the KGF-2 polypeptide encoded by the deposited cDNA or for the FGF-12 polypeptide of FIG. 3 (SEQ ID NO:6) may include: only the coding sequence for the polypeptide; the coding sequence for the polypeptide and additional coding sequence such as a leader or secretary sequence or a proprotein sequence; the coding sequence for the polypeptide (and optionally additional coding sequence) and non-coding sequence, such as intron or non-coding sequence 5′ and/or 3′ of the coding sequence for the predicted KGF-2 polypeptide.

Thus, the term “polynucleotide encoding a polypeptide” encompasses a polynucleotide which includes only coding sequence for the polypeptide as well as a polynucleotide which includes additional coding and/or non-coding sequence.

The present invention further relates to variants of the hereinabove described polynucleotides which encode for fragments, analogs and derivatives of the polypeptide having the deduced amino acid sequence of FIG. 1 (SEQ ID NO:2) or the polypeptide encoded by the cDNA of the deposited clone. The variant of the polynucleotide may be a naturally occurring allelic variant of the polynucleotide or a nonnaturally occurring variant of the polynucleotide.

Thus, the present invention includes using polynucleotides encoding the same polypeptide as shown in FIG. 1 (SEQ ID NO:2) or the same KGF-2 polypeptide encoded by the cDNA of the deposited clone as well as variants of such polynucleotides which variants encode for a fragment, derivative or analog of the polypeptide of FIG. 1 (SEQ ID NO:2) or the polypeptide encoded by the cDNA of the deposited clone. The present invention also includes using polynucleotides encoding the same polypeptide as shown in FIG. 3 (SEQ ID NO:6) as well as variants of such polynucleotides which variants encode for a fragment, derivative or analog of the polypeptide of FIG. 3 (SEQ ID NO:6). Such nucleotide variants include deletion variants, substitution variants and addition or insertion variants.

As hereinabove indicated, the polynucleotide may have a coding sequence which is a naturally occurring allelic variant of the coding sequence shown in FIG. 1 (SEQ ID NO:1) or of the coding sequence of the deposited clone or of FIG. 3 (SEQ ID NO:5). As known in the art, an allelic variant is an alternate form of a polynucleotide sequence which may have a substitution, deletion or addition of one or more nucleotides, which does not substantially alter the function of the encode polypeptide.

The present invention also includes polynucleotides, wherein the coding sequence for the polypeptide may be fused in the same reading frame to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and may have the leader sequence cleaved by the host cell to form the mature form of the polypeptide. The polynucleotides may also encode for proprotein which is the protein plus additional 5′ amino acid residues. A mature protein having a prosequence is a proprotein and is an inactive form of the protein. Once the prosequence is cleaved an active mature protein remains.

Thus, for example, the polynucleotide useful in the methods of the present invention may encode for a mature KGF-2 or FGF-12 protein alone, or for a protein having a prosequence or for a protein having both prosequence and a presequence (leader sequence).

The polynucleotides useful in the present invention may also have the coding sequence fused in frame to a marker sequence which allows for purification of the polypeptide of the present invention. The marker sequence may be a hexahistidine tag supplied by a pQE-9 vector to provide for purification of the polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I. et al. Cell 37:767 (1984)).

The term “gene” means the segment of DNA involved in producing a polypeptide chain; it includes regions preceding and following the coding region (leader and trailer) as well as intervening sequences (introns) between individual coding segments (exons).

Further embodiments of the invention include isolated nucleic acid molecules comprising a polynucleotide having a nucleotide sequence at least 90% identical, and more preferably at least 95%, 97%, 98% or 99% identical to (a) a nucleotide sequence encoding the full-length KGF-2 polypeptide having the complete amino acid sequence in FIG. 1 (SEQ ID NO:2), including the predicted leader sequence; (b) a nucleotide sequence encoding the mature KGF-2 polypeptide (full-length polypeptide with the leader removed) having the amino acid sequence at positions about 36 or 37 to 208 in FIG. 1 (SEQ ID NO:2); (c) a nucleotide sequence encoding the full-length KGF-2 polypeptide having the complete amino acid sequence including the leader encoded by the cDNA clone contained in ATCC Deposit No. 75977; (d) a nucleotide sequence encoding the mature KGF-2 polypeptide having the amino acid sequence encoded by the cDNA clone contained in ATCC Deposit No. 75977; (e) a nucleotide sequence encoding the FGF-12 polypeptide having the complete amino acid sequence in FIG. 3 (SEQ ID NO:6); (f) a nucleotide sequence of FGF-12 in FIG. 3 (SEQ ID NO:5); (g) a nucleotide sequence encoding any of the KGF-2 analogs or deletion mutants described below; or (h) a nucleotide sequence complementary to any of the nucleotide sequences in (a), (b), (c), (d), (e), (f) or (g).

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the KGF-2 or FGF-12 polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown of SEQ ID NO:1 or 5 or any fragment specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245.) In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identify are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

The KGF-2 or FGF-12 variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. KGF-2 polynucleotide variants can be produced for a variety of reasons, e.g., to optimize codon expression for a particular host (change codons in the human mRNA to those preferred by a bacterial host such as E. coli).

Alternatively, whether any particular nucleic acid molecule is at least 90%, 95%, 96%, 97%, 98% or 99% identical to, for instance, the nucleotide sequence shown in FIG. 1 (SEQ ID NO:1) or to the nucleotides sequence of the deposited cDNA clones or shown in FIG. 3 (SEQ ID NO:4) may also be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

Preferred, are nucleic acid molecules having sequences at least 90%, 95%, 96%, 97%, 98% or 99% identical to the nucleic acid sequence shown in FIG. 1 (SEQ ID NO:1) or to the nucleic acid sequence of the deposited cDNA which do, in fact, encode a polypeptide having KGF-2 protein activity, or to the nucleic acid sequence shown in FIG. 3 (SEQ ID NO:5) which do, in fact, encode a polypeptide having FGF-12 activity. By “a polypeptide having KGF-2 activity” is intended polypeptides exhibiting activity similar, but not necessarily identical, to an activity of the wild-type KGF-2 protein or an activity that is enhanced over that of the wild-type KGF-2 protein (either the full-length protein or, preferably, the mature protein), as measured in a particular biological assay. By “a polypeptide having FGF-12 activity” is intended polypeptides exhibiting activity similar, but not necessarily identical, to an activity of the wild-type FGF-12 protein or an activity that is enhanced over that of the wild-type FGF-12 protein, as measured in a particular biological assay.

By “KGF-2 polypeptide” is meant the full length or mature KGF-2 polypeptide described herein, or any deletion and/or substitution mutant thereof.

KGF-2 stimulates the proliferation of epidermal keratinocytes but not mesenchymal cells such as fibroblasts. Thus, “a polypeptide having KGF-2 protein activity” includes polypeptides that exhibit the KGF-2 activity in the keratinocyte proliferation assay set forth in Example 1 below and will bind to the FGF receptor isoforms 1-iiib and 2-iiib. Although the degree of activity need not be identical to that of the KGF-2 protein, preferably, “a polypeptide having KGF-2 protein activity” will exhibit substantially similar activity as compared to the KGF-2 protein (i.e., the candidate polypeptide will exhibit greater activity or not more than about tenfold less and, preferably, not more than about twofold less activity relative to the reference KGF-2 protein).

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA or the nucleic acid sequence shown in FIG. 1 (SEQ ID NO:1) or FIG. 3 (SEQ ID NO:5) will encode a polypeptide “having KGF-2 protein activity” or “having FGF-12 protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having KGF-2 or FGF-12 protein activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections or screens to identify sequences that maintain functionality. As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, J. U. et al., supra, and the references cited therein.

The deposit(s) referred to herein will be maintained under the terms of the Budapest Treaty on the International Recognition of the Deposit of Micro-organisms for purposes of Patent Procedure. These deposits are provided merely as convenience to those of skill in the art and are not an admission that a deposit is required under 35 U.S.C. §112. The sequence of the polynucleotides contained in the deposited materials, as well as the amino acid sequence of the polypeptides encoded thereby, are incorporated herein by reference and are controlling in the event of any conflict with any description of sequences herein. A license may be required to make, use or sell the deposited materials, and no such license is hereby granted.

The present invention further relates to producing a KGF-2 polypeptide which has the deduced amino acid sequence of FIG. 1 (SEQ ID NO:2) or which has the amino acid sequence encoded by the deposited cDNA, as well as fragments, analogs and derivatives of such polypeptide. The present invention also relates to producing a FGF-12 polypeptide which has the deduced amino acid sequence of FIG. 3 (SEQ ID NO:6) as well as fragments, analogs and derivatives of such polypeptide.

The terms “fragment,” “derivative” and “analog” when referring to the polypeptide, of FIG. 1 (SEQ ID NO:2) or that encoded by the deposited cDNA or of FIG. 3 (SEQ ID NO:6), means a polypeptide which retains essentially the same biological function or activity as such polypeptide. Thus, an analog includes a protein which can be activated by cleavage of the protein portion to produce an active polypeptide.

The fragment, derivative or analog of the polypeptide of FIG. 1 (SEQ ID NO:2) or that encoded by the deposited cDNA or of FIG. 3 (SEQ ID NO:6) may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which the additional amino acids are fused to the polypeptide, such as a leader or secretary sequence or a sequence which is employed for purification of the polypeptide or a protein sequence. Such fragments, derivatives and analogs are deemed to be within the scope of those skilled in the art from the teachings herein.

By a fragment of an isolated the KGF-2 polypeptide, for example, encoded by the deposited cDNA, the polypeptide sequence encoded by the deposited cDNA, the polypeptide sequence depicted in FIG. 1 (SEQ ID NO:2), is intended to encompass polypeptide fragments contained in SEQ ID NO:2 or encoded by the cDNA contained in the deposited clone. Protein fragments may be “free-standing,” or comprised within a larger polypeptide of which the fragment forms a part or region, most preferably as a single continuous region. Polypeptide fragments can be at least 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Even if deletion of one or more amino acids from the N-terminus of a protein results in modification of loss of one or more biological functions of the protein, other functional activities (e.g., biological activities, ability to multimerize, ability to bind KGF-2 ligand) may still be retained. For example, the ability of shortened KGF-2 mutants to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that an KGF-2 mutant with a large number of deleted N-terminal amino acid residues may retain some biological or immunogenic activities. In fact, peptides composed of as few as six KGF-2 amino acid residues may often evoke an immune response.

Accordingly, polypeptide fragments include the secreted KGF-2 protein as well as the mature form. Further preferred polypeptide fragments include the secreted KGF-2 protein or the mature form having a continuous series of deleted residues from the amino or the carboxy terminus, or both. For example, any number of amino acids, ranging from 1-60, can be deleted from the amino terminus of either the secreted KGF-2 polypeptide or the mature form. Similarly, any number of amino acids, ranging from 1-30, can be deleted from the carboxy terminus of the secreted KGF-2 protein or mature form. Furthermore, any combination of the above amino and carboxy terminus deletions are preferred. Similarly, polynucleotide fragments encoding these KGF-2 polypeptide fragments are also preferred.

Particularly, N-terminal deletions of the KGF-2 polypeptide can be described by the general formula m−208, where m is an integer from 2 to 207, where m corresponds to the position of the amino acid residue identified in SEQ ID NO:2. More in particular, the invention provides polynucleotides encoding polypeptides comprising, or alternatively consisting of, the amino acid sequence of residues of W-2 to S-208; K-3 to S-208; W-4 to S-208; I-5 to S-208; L-6 to S-208; T-7 to S-208; H-8 to S-208; C-9 to S-208; A-10 to S-208; S-11 to S-208; A-12 to S-208; F-13 to S-208; P-14 to S-208; H-15 to S-208; L-16 to S-208; P-17 to S-208; G-18 to S-208; C-19 to S-208; C-20 to S-208; C-21 to S-208; C-22 to S-208; C-23 to S-208; F-24 to S-208; L-25 to S-208; L-26 to S-208; L-27 to S-208; F-28 to S-208; L-29 to S-208; V-30 to S-208; S-31 to S-208; S-32 to S-208; V-33 to S-208; P-34 to S-208; V-35 to S-208; T-36 to S-208; C-37 to S-208; Q-38 to S-208; A-39 to S-208; L-40 to S-208; G-41 to S-208; Q-42 to S-208; D-43 to S-208; M-44 to S-208; V-45 to S-208; S-46 to S-208; P-47 to S-208; E-48 to S-208; A-49 to S-208; T-50 to S-208; N-51 to S-208; S-52 to S-208; S-53 to S-208; S-54 to S-208; S-55 to S-208; S-56 to S-208; F-57 to S-208; S-58 to S-208; S-59 to S-208; P-60 to S-208; S-61 to S-208; S-62 to S-208; A-63 to S-208; G-64 to S-208; R-65 to S-208; H-66 to S-208; V-67 to S-208; and R-68 to S-208.

Also as mentioned above, even if deletion of one or more amino acids from the C-terminus of a protein results in modification of loss of one or more biological functions of the protein, other functional activities (e.g., biological activities, ability to multimerize, ability to bind KGF-2 ligand) may still be retained. For example the ability of the shortened KGF-2 mutant to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that an KGF-2 mutant with a large number of deleted C-terminal amino acid residues may retain some biological or immunogenic activities. In fact, peptides composed of as few as six KGF-2 amino acid residues may often evoke an immune response.

Accordingly, the present invention further provides polypeptides having one or more residues deleted from the carboxy terminus of the amino acid sequence of the KGF-2 polypeptide shown in FIG. 1 (SEQ ID NO:2), as described by the general formula 1−n, where n is an integer from 2 to 207, where n corresponds to the position of amino acid residue identified in SEQ ID NO:2. More in particular, the invention provides polynucleotides encoding polypeptides comprising, or alternatively consisting of, the amino acid sequence of residues M-1 to H-207; M-1 to V-206; M-1 to V-205; M-1 to M-204; M-1 to P-203; M-1 to L-202; M-1 to F-201; M-1 to H-200; M-1 to A-199; M-1 to S-198; M-1 to T-197; M-1 to N-196; M-1 to K-195; M-1 to R-194; M-1 to R-193; M-1 to T-192; M-1 to K-191; M-1 to Q-190; M-1 to G-189; M-1 to R-188; M-1 to R-187; M-1 to P-186; M-1 to A-185; M-1 to G-184; M-1 to K-183; M-1 to G-182; M-1 to N-181; M-1 to L-180; M-1 to A-179; M-1 to V-178; M-1 to Y-177; M-1 to M-176; M-1 to Q-175; M-1 to R-174; M-1 to G-173; M-1 to N-172; M-1 to H-171; M-1 to Q-170; M-1 to W-169; M-1 to N-168; M-1 to F-167; M-1 to S-166; M-1 to A-165; M-1 to Y-164; M-1 to T-163; M-1 to N-162; M-1 to Y-161; M-1 to G-160; M-1 to N-159; M-1 to E-158; M-1 to E-157; M-1 to I-156; M-1 to R-155; M-1 to E-154; M-1 to K-153; M-1 to L-152; M-1 to K-151; M-1 to C-150; M-1 to D-149; M-1 to N-148; M-1 to N-147; M-1 to F-146; M-1 to E-145; M-1 to K-144; M-1 to S-143; M-1 to G-142; M-1 to Y-141; M-1 to L-140; M-1 to K-139; M-1 to G-138; M-1 to K-137; M-1 to K-136; M-1 to N-135; M-1 to M-134; M-1 to A-133; M-1 to L-132; M-1 to Y-131; M-1 to Y-130; M-1 to N-129; M-1 to S-128; M-1 to N-127; M-1 to I-126;M-1 to A-125; M-1 to K-124; M-1 to V-123; M-1 to A-122; M-1 to V-121; M-1 to V-120; M-1 to G-119; M-1 to I-118; M-1 to E-117; M-1 to V-116; M-1 to S-115; M-1 to T-114; M-1 to I-113; M-1 to E-112; M-1 to L-111; M-1 to I-110; M-1 to S-109; M-1 to Y-108; M-1 to P-107; M-1 to C-106; M-1 to N-105; M-1 to E-104; M-1 to K-103; M-1 to K-102; M-1 to T-101; M-1 to G-100; M-1 to S-99; M-1 to V-98; M-1 to K-97; M-1 to G-96; M-1 to N-95; M-1 to K-94; M-1 to E-93; M-1 to I-92; M-1 to K-91; M-1 to L-90; M-1 to F-89; M-1 to Y-88; M-1 to K-87; M-1 to T-86; M-1 to F-85; M-1 to S-84; M-1 to F-83; M-1 to L-82; M-1 to K-81; M-1 to R-80; M-1 to W-79; M-1 to R-78; M-1 to V-77; M-1 to D-76; M-1 to G-75; M-1 to Q-74; M-1 to L-73; M-1 to H-72; M-1 to N-71; M-1 to Y-70; and M-1 to S-69.

Likewise, C-terminal deletions of the KGF-2 polypeptide of the invention shown as SEQ ID NO:2 include polypeptides comprising the amino acid sequence of residues: A-63 to H-207; A-63 to V-206; A-63 to V-205; A-63 to M-204; A-63 to P-203; A-63 to L-202; A-63 to F-201; A-63 to H-200; A-63 to A-199; A-63 to S-198; A-63 to T-197; A-63 to N-196; A-63 to K-195; A-63 to R-194; A-63 to R-193; A-63 to T-192; A-63 to K-191; A-63 to Q-190; A-63 to G-189; A-63 to R-188; A-63 to R-187; A-63 to P-186; A-63 to A-185; A-63 to G-184; A-63 to K-183; A-63 to G-182; A-63 to N-181; A-63 to L-180; A-63 to A-179; A-63 to V-178; A-63 to Y-177; A-63 to M-176; A-63 to Q-175; A-63 to R-174; A-63 to G-173; A-63 to N-172; A-63 to H-171; A-63 to Q-170; A-63 to W-169; A-63 to N-168; A-63 to F-167; A-63 to S-166; A-63 to A-165; A-63 to Y-164; A-63 to T-163; A-63 to N-162; A-63 to Y-161; A-63 to G-160; A-63 to N-159; A-63 to E-158; A-63 to E-157; A-63 to I-156; A-63 to R-155; A-63 to E-154; A-63 to K-153; A-63 to L-152; A-63 to K-151; A-63 to C-150; A-63 to D-149; A-63 to N-148; A-63 to N-147; A-63 to F-146; A-63 to E-145; A-63 to K-144; A-63 to S-143; A-63 to G-142; A-63 to Y-141; A-63 to L-140; A-63 to K-139; A-63 to G-138; A-63 to K-137; A-63 to K-136; A-63 to N-135; A-63 to M-134; A-63 to A-133; A-63 to L-132; A-63 to Y-131; A-63 to Y-130; A-63 to N-129; A-63 to S-128; A-63 to N-127; A-63 to I-126; A-63 to A-125; A-63 to K-124; A-63 to V-123; A-63 to A-122; A-63 to V-121; A-63 to V-120; A-63 to G-119; A-63 to I-118; A-63 to E-117; A-63 to V-116; A-63 to S-115; A-63 to T-114; A-63 to I-113; A-63 to E-112; A-63 to L-111; A-63 to I-110; A-63 to S-109; A-63 to Y-108; A-63 to P-107; A-63 to C-106; A-63 to N-105; A-63 to E-104; A-63 to K-103; A-63 to K-102; A-63 to T-101; A-63 to G-100; A-63 to S-99; A-63 to V-98; A-63 to K-97; A-63 to G-96; A-63 to N-95; A-63 to K-94; A-63 to E-93; A-63 to I-92; A-63 to K-91; A-63 to L-90; A-63 to F-89; A-63 to Y-88; A-63 to K-87; A-63 to T-86; A-63 to F-85; A-63 to S-84; A-63 to F-83; A-63 to L-82; A-63 to K-81; A-63 to R-80; A-63 to W-79; A-63 to R-78; A-63 to V-77; A-63 to D-76; and A-63 to G-75 of SEQ ID NO:2.

In addition, any of the above listed N- or C-terminal deletions can be combined to produce a N- and C-terminal deleted KGF-2 polypeptide. The invention also provides polypeptides having one or more amino acids deleted from both the amino and the carboxyl termini, which may be described generally as having residues m-n of SEQ ID NO:2, where n and m are integers as described above. In addition, N- or C-terminal deletion mutants may also contain site specific amino acid substitutions. Polynucleotides encoding these polypeptides are also encompassed by the invention.

Particularly preferred are fragments comprising or consisting of: A39-S208; A63-N162; and M35-N162.

The terms “peptide” and “oligopeptide” are considered synonymous (as is commonly recognized) and each term can be used interchangeably as the context requires to indicate a chain of at least two amino acids coupled by peptidyl linkages. The word “polypeptide” is used herein for chains containing more than ten amino acid residues. All oligopeptide and polypeptide formulas or sequences herein are written from left to right and in the direction from amino terminus to carboxy terminus.

It will be recognized in the art that some amino acid sequences of the polypeptide can be varied without significant effect of the structure or function of the protein. If such differences in sequence are contemplated, it should be remembered that there will be critical areas on the protein which determine activity. In general, it is possible to replace residues which form the tertiary structure, provided that residues performing a similar function are used. In other instances, the type of residue may be completely unimportant if the alteration occurs at a non-critical region of the protein.

Thus, the invention further includes variations of the KGF-2 or FGF-12 polypeptide which show substantial KGF-2 or FGF-12 polypeptide activity or which include regions of KGF-2 or FGF-12 protein such as the protein portions discussed below. Such mutants include deletions, insertions, inversions, repeats, and type substitutions (for example, substituting one hydrophilic residue for another, but not strongly hydrophilic for strongly hydrophobic as a rule). Small changes or such “neutral” amino acid substitutions will generally have little effect on activity.

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

As indicated in detail above, further guidance concerning which amino acid changes are likely to be phenotypically silent (i.e., are not likely to have a significant deleterious effect on a function) can be found in Bowie, J. U., et al., “Deciphering the Message in Protein Sequences: Tolerance to Amino Acid Substitutions,” Science 247:1306-1310 (1990).

The polypeptides and polynucleotides of the present invention are preferably provided in an isolated form, and preferably are purified to homogeneity.

The polypeptides produced by the methods of the present invention are preferably in an isolated form. By “isolated polypeptide” is intended a polypeptide removed from its native environment. Thus, a polypeptide produced and/or contained within a recombinant host cell is considered isolated for purposes of the present invention. Also intended are polypeptides that have been purified, partially or substantially, from a recombinant host cell or a native source.

The polypeptides of the present invention include the polypeptide of SEQ ID NO:2 (in particular the mature KGF-2 polypeptide) as well as polypeptides which have at least 90%, 95%, 96%, 97%, 98%, 99% similarity (more preferably at least 90%, 95%, 96%, 97%, 98%, 99% identity) to the polypeptide of SEQ ID NO:2 or SEQ ID NO:6 and also include portions of such polypeptides with such portion of the polypeptide (such as the deletion mutants described below) generally containing at least 30 amino acids and more preferably at least 50 amino acids.

As known in the art “similarity” between two polypeptides is determined by comparing the amino acid sequence and its conserved amino acid substitutes of one polypeptide to the sequence of a second polypeptide.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2: 482-489, 1981) to find the best segment of similarity between two sequences.

By a polypeptide having an amino acid sequence at least, for example, 95% “identical” to a reference amino acid sequence of a KGF-2 or FGF-12 polypeptide is intended that the amino acid sequence of the polypeptide is identical to the reference sequence except that the polypeptide sequence may include up to five amino acid alterations per each 100 amino acids of the reference amino acid of the KGF-2 or FGF-12 polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in SEQ ID NO:2 or to the amino acid sequence encoded by deposited DNA clone or the amino acid sequence of SEQ ID NO:6 can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of the present invention.

Alternatively, whether any particular polypeptide is at least 90%, 95%, 96%, 97%, 98% or 99% identical to, for instance, the amino acid sequence shown in FIG. 1 (SEQ ID NO:2) or to the amino acid sequence encoded by deposited cDNA clone or FIG. 3 (SEQ ID NO:6) may also be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In accordance with the present invention, novel variants of KGF-2 are also described. These can be produced by deleting or substituting one or more amino acids of KGF-2. Natural mutations are called allelic variations. Allelic variations can be silent (no change in the encoded polypeptide) or may have altered amino acid sequence.

A further aspect of the present invention also includes the substitution of amino acids. Native mature KGF-2 contains 44 charged residues, 32 of which carry a positive charge. Depending on the location of such residues in the protein's three dimensional structure, substitution of one or more of these clustered residues with amino acids carrying a negative charge or a neutral charge may alter the electrostatic interactions of adjacent residues and may be useful to achieve increased stability and reduced aggregation of the protein. Aggregation of proteins cannot only result in a loss of activity but be problematic when preparing pharmaceutical formulations, because they can be immunogenic (Pinckard et al., Clin. Exp. Immunol. 2:331-340 (1967), Robbins et al., Diabetes 36: 838-845 (1987), Cleland et al., Crit. Rev. Therapeutic Drug Carrier Systems 10: 307-377 (1993)). Any modification should give consideration to minimizing charge repulsion in the tertiary structure of the protein molecule. Thus, of special interest are substitutions of charged amino acid with another charge and with neutral or negatively charged amino acids. The latter results in proteins with a reduced positive charge to improve the characteristics of KGF-2. Such improvements include increased stability and reduced aggregation of the analog as compared to the native KGF-2 protein.

The replacement of amino acids can also change the selectivity of binding to cell surface receptors. Ostade et al., Nature 361: 266-268 (1993), described certain TNF alpha mutations resulting in selective binding of TNF alpha to only one of the two known TNF receptors.

A further embodiment of the invention relates to producing a polypeptide which comprises the amino acid sequence of a KGF-2 or FGF-12 polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a peptide or polypeptide to have an amino acid sequence which comprises the amino acid sequence of a KGF-2 or FGF-12 polypeptide, which contains at least one, but not more than 20, 19, 18, 17, 16, 15, 14, 13, 12, 11, 10, 9, 8, 7, 6, 5, 4, 3, 2 or 1 amino acid substitutions. In specific embodiments, the number of additions, substitutions, and/or deletions in the amino acid sequence of FIG. 1 or FIG. 3 or fragments thereof, is 1-5, 5-10, 5-25, 5-50, 10-50 or 50-150, conservative amino acid substitutions are preferable.

KGF-2 molecules may include one or more amino acid substitutions, deletions or additions, either from natural mutation or human manipulation. The mutations can be made in full-length KGF-2, mature KGF-2, any other appropriate fragments of KGF-2, for example, A63-S208. Examples of some preferred mutations are: Ala (49) Gln, Asn (51) Ala, Ser (54) Val, Ala (63) Pro, Gly (64) Glu, Val (67) Thr, Trp (79) Val, Arg (80) Lys, Lys (87) Arg, Tyr (88) Trp, Phe (89) Tyr, Lys (91) Arg, Ser (99) Lys, Lys (102) Gln, Lys 103(Glu), Glu (104) Met, Asn (105) Lys, Pro (107) Asn, Ser (109) Asn, Leu (111) Met, Thr (114) Arg, Glu(117) Ala, Val (120) Ile, Val (123) Ile, Ala (125) Gly, Ile (126) Val, Asn (127) Glu, Asn (127) Gln, Tyr (130) Phe, Met (134) Thr, Lys (136) Glu, Lys (137) Glu, Gly (142) Ala, Ser (143) Lys, Phe (146) Ser, Asn (148) Glu, Lys (151) Asn, Leu (152) Phe, Glu (154) Gly, Glu (154) Asp, Arg (155) Leu, Glu (157) Leu, Gly (160) His, Phe (167) Ala, Asn (168) Lys, Gln (170) Thr, Arg (174) Gly, Tyr (177) Phe, Gly (182) Gln, Ala (185) Val, Ala (185) Leu, Ala (185) Ile, Arg (187) Gln (190) Lys, Lys (195) Glu, Thr (197) Lys, Ser (198) Thr, Arg (194) Glu, Arg (194) Gln, Lys (191) Glu, Lys (191) Gln, Arg (188) Glu, Arg (188) Gln, Lys (183) Glu, Arg (187) Ala, Arg (188) Ala, Arg 174 (Ala), Lys (183) Ala, Lys (144) Ala, Lys (151) Ala, Lys (153) Ala, Lys (136) Ala, Lys (137) Ala, and Lys (139) Ala.

By the designation, for example, Ala (63) Pro is intended that the Ala at position 63 of FIG. 1 (SEQ ID NO:2) is replaced by Pro.

Additionally, the following mutants are particularly preferred: KGF-2Δ28 with a point mutation at R68G; KGF-2Δ28 with a point mutation at R68S; KGF-2Δ28 with a point mutation at R68A; KGF-2Δ28 with point mutations at R78A, R80A and K81A; KGF-2Δ28 with point mutations at K81A, K87A and K91A; KGF-2Δ28 with point mutations at R78A, R80A, K81A, K87A and K91A; KGF-2Δ28 with point mutations at K136A, K137A, K139A and K144A; KGF-2Δ28 with point mutations at K151A, K153A and K155A; KGF-2Δ28 with point mutations at R68G, R78A, R80A, and K81A; KGF-2Δ28 with point mutations at R68G, K81A, K87A and K91A; KGF-2Δ28 with point mutations at R68G, R78A, R80A, K81A, K87A and K91A; KGF-2Δ28 with point mutations, at R68G, K136A, K137A, K139A, and K144A; A63-208 with point mutations at R68G, K151A, K153A, and R155A; KGF-2Δ28 with point mutations at R68S, R78A, R80A, and K81A; KGF-2Δ28 with point mutations at R68S, K81A, R87A and K91A; KGF-2Δ28 with point mutations at R68S, R78A, R80A, K81A, K87A and K91A; KGF-2Δ28 with point mutations at R68S, K136A, K137A, K139A, and K144A; A63-208 with point mutations at R68S, K151A, K153A, and R155A; KGF-2Δ28 with point mutations at R68A, R78A, R80A and K81A; KGF-2Δ28 with point mutations at R68A, K81A, K87A, and K91A; KGF-2Δ28 with point mutations at R68A, R78A, R80A, K81A, K87A, and K91A; KGF-2Δ28 with point mutations at R68A, K136A, K137A, K139A and K144A; and KGF-2Δ28 with point mutations at R68A, K151A, K153A and R155A. Also preferred are: KGF-2Δ28 with the positively charged residues between and including R68 to K91 are replaced with alanine; KGF-2Δ28 with the positively charged residues between and including R68 to K91 replaced with neutral residues, such as G, S and/or A; KGF-2Δ28 with the positively charged residues between and including R68 to K91 replaced with negatively charged acidic residues, such as D and/or E; KGF-2Δ28 with point mutations at R174A and K183A; KGF-2Δ28 with point mutations at R187A and R188A; and KGF-2Δ28 with a point mutation at R188E, K191E, K149E, K183Q, or K183E.

All of the above point mutations may also be made in the full length KGF-2, the mature KGF-2, or any other fragment of KGF-2 described herein. By the designation, for sample, R188E is intended that the Arginine at position 188 is replaced with a Glutamic Acid.

In addition site directed mutations may be made at each amino acids of KGF-2, preferably between amino acids A63 to E93. Each amino acid can be replaced by any of the other 19 remaining amino acids. For example preferred mutations include: A63 replaced with C, D, E, F, G, H, I, K, L, M, N, P, Q, R, S, T, V, W, or Y; G64 replaced with A, C, D, E, F, H, I, K, L, M, N, P, Q, R, S, T, V, W, or Y; R65 replaced with A, C, D, E, F, G, H, I, K, L, M, N, P, Q, S, T, V, W, or Y; H66 replaced with A, C, D, E, F, G, I, K, L, M, N, P, Q, R, S, T, V, W, or Y; V67 replaced with A, C, D, E, F, G, H, I, K, L, M, N, P, Q, R, S, T, W, or Y; R68 replaced with A, C, D, E, F, G, H, I, K, L, M, N, P, Q, S, T, V, W, or Y; S69 replaced with A, C, D, E, F, G, H, I, K, L, M, N, P, Q, R, T, V, W, or Y; Y70 replaced with A, C, D, E, F, G, H, I, K, L, M, N, P, Q, R, S, T, V, or W; N71 replaced with A, C, D, E, F, G, H, I, K, L, M, P, Q, R, S, T, V, W, or Y; H72 replaced with A, C, D, E, F, G, I, K, L, M, N, P, Q, R, S, T, V, W, or Y; L73 replaced with A, C, D, E, F, G, H, I, K, M, N, P, Q, R, S, T, V, W, or Y; Q74 replaced with A, C, D, E, F, G, H, I, K, L, M, N, P, R, S, T, V, W, or Y; G75 replaced with A, C, D, E, F, H, I, K, L, M, N, P, Q, R, S, T, V, W, or Y; D76 replaced with A, C, E, F, G, H, I, K, L, M, N, P, Q, R, S, T, V, W, or Y; V77 replaced with A, C, D, E, F, G, H, I, K, L, M, N, P, Q, R, S, T, W, or Y; R78 replaced with A, C, D, E, F, G, H, I, K, L, M, N, P, Q, S, T, V, W, or Y; W79 replaced with A, C, D, E, F, G, H, I, K, L, M, N, P, Q, R, S, T, V, or Y; R80 replaced with A, C, D, E, F, G, H, I, K, L, M, N, P, Q, S, T, V, W, or Y; K81 replaced with A, C, D, E, F, G, H, I, L, M, N, P, Q, R, S, T, V, W, or Y; L82 replaced with A, C, D, E, F, G, H, I, K, M, N, P, Q, R, S, T, V, W, or Y; F83 replaced with A, C, D, E, G, H, I, K, L, M, N, P, Q, R, S, T, V, W, or Y; S84 replaced with A, C, D, E, F, G, H, I, K, L, M, N, P, Q, R, T, V, W, or Y; F85 replaced with A, C, D, E, G, H, I, K, L, M, N, P, Q, R, S, T, V, W, or Y; T86 replaced with A, C, D, E, F, G, H, I, K, L, M, N, P, Q, R, S, V, W, or Y; K87 replaced with A, C, D, E, F, G, H, I, L, M, N, P, Q, R, S, T, V, W, or Y; Y88 replaced with A, C, D, E, F, G, H, I, K, L, M, N, P, Q, R, S, T, V, or W; F89 replaced with A, C, D, E, G, H, I, K, L, M, N, P, Q, R, S, T, V, W, or Y; L90 replaced with A, C, D, E, F, G, H, I, K, M, N, P, Q, R, S, T, V, W, or Y; K91 replaced with A, C, D, E, F, G, H, I, L, M, N, P, Q, R, S, T, V, W, or Y; I92 replaced with A, C, D, E, F, G, H, K, L, M, N, P, Q, R, S, T, V, W, or Y; and/or E93 replaced with A, C, D, F, G, H, I, K, L, M, N, P, Q, R, S, T, V, W, or Y.

More than one amino acid (e.g. 2, 3, 4, 5, 6, 7, 8, 9 and 10) can be replaced in this region (A63 to E93) with other amino acids. The resulting constructs can be screened for loss of heparin binding, loss of KGF-2 activity, and/or loss of enzymatic cleavage between amino acids R68 and S69.

Preferred mutations are located at amino acid positions R68 and S69 in N-terminal deletion constructs M1, T36, C37 and A63, as well as mutations in the heparin binding domain, of all of the above listed N-terminal mutants, especially T36, C37, and A63. The heparin binding domain is between Arg174 and Lys 183. Preferred Arg68 mutants replace the arginine with Gly, Ser or Ala; preferred Arg187 mutants replace the arginine with alanine.

Two ways in which mutations can be made is either by site directed mutagenesis or accelerated mutagenesis (Kuchner and Arnold, Tibtech 5:523-530 (1997); Crameri et al., Nature (1998); and Christians et al., Nature Biotechnology 17:259264 (1999)). These methods are well known in the art.

Changes are preferably of minor nature, such as conservative amino acid substitutions that do not significantly affect the folding or activity of the protein. Examples of conservative amino acid substitutions known to those skilled in the art are set forth below:

| | |
|---|---|
| Aromatic: | phenylalanine |
| | tryptophan |
| | tyrosine |
| Hydrophobic: | leucine |
| | isoleucine |
| | valine |
| Polar: | glutamine |
| | asparagine |
| Basic: | arginine |
| | lysine |
| | histidine |
| Acidic: | aspartic acid |
| | glutamic acid |
| Small: | alanine |
| | serine |
| | threonine |
| | methionine |
| | glycine |

Of course, the number of amino acid substitutions a skilled artisan would make depends on many factors, including those described above. Generally speaking, the number of substitutions for any given KGF-2 or FGF-12 polypeptide will not be more than 50, 40, 30, 20, 10, 5, or 3, depending on the objective. For example, a number of substitutions that can be made in the C-terminus of KGF-2 to improve stability are described above.

Particularly preferred are KGF-2 molecules with conservative amino acid substitutions, including: M1 replaced with A, G, I, L, S, T, or V; W2 replaced with F, or Y; K3 replaced with H, or R; W4 replaced with F, or Y; I5 replaced with A, G, L, S, T, M, or V; L6 replaced with A, G, I, S, T, M, or V; T7 replaced with A, G, I, L, S, M, or V; H8 replaced with K, or R; A10 replaced with G, I, L, S, T, M, or V; S11 replaced with A, G, I, L, T, M, or V; A12 replaced with G, I, L, S, T, M, or V; F13 replaced with W, or Y; H15 replaced with K, or R; L16 replaced with A, G, I, S, T, M, or V; G18 replaced with A, I, L, S, T, M, or V; F24 replaced with W, or Y; L25 replaced with A, G, I, S, T, M, or V; L26 replaced with A, G, I, S, T, M, or V; L27 replaced with A, G, I, S, T, M, or V; F28 replaced with W, or Y; L29 replaced with A, G, I, S, T, M, or V; V30 replaced with A, G, I, L, S, T, or M; S31 replaced with A, G, I, L, T, M, or V; S32 replaced with A, G, I, L, T, M, or V; V33 replaced with A, G, I, L, S, T, or M; V35 replaced with A, G, I, L, S, T, or M; T36 replaced with A, G, I, L, S, M, or V; Q38 replaced with N; A39 replaced with G, I, L, S, T, M, or V; L40 replaced with A, G, I, S, T, M, or V; G41 replaced with A, I, L, S, T, M, or V; Q42 replaced with N; D43 replaced with E; M44 replaced with A, G, I, L, S, T, or V; V45 replaced with A, G, I, L, S, T, or M; S46 replaced with A, G, I, L, T, M, or V; E48 replaced with D; A49 replaced with G, I, L, S, T, M, or V; T50 replaced with A, G, I, L, S, M, or V; N51 replaced with Q; S52 replaced with A, G, I, L, T, M, or V; S53 replaced with A, G, I, L, T, M, or V; S54 replaced with A, G, I, L, T, M, or V; S55 replaced with A, G, I, L, T, M, or V; S56 replaced with A, G, I, L, T, M, or V; F57 replaced with W, or Y; S58 replaced with A, G, I, L, T, M, or V; S59 replaced with A, G, I, L, T, M, or V; S61 replaced with A, G, I, L, T, M, or V; S62 replaced with A, G, I, L, T, M, or V; A63 replaced with G, I, L, S, T, M, or V; G64 replaced with A, I, L, S, T, M, or V; R65 replaced with H, or K; H66 replaced with K, or R; V67 replaced with A, G, I, L, S, T, or M; R68 replaced with H, or K; S69 replaced with A, G, I, L, T, M, or V; Y70 replaced with F, or W; N71 replaced with Q; H72 replaced with K, or R; L73 replaced with A, G, I, S, T, M, or V; Q74 replaced with N; G75 replaced with A, I, L, S, T, M, or V; D76 replaced with E; V77 replaced with A, G, I, L, S, T, or M; R78 replaced with H, or K; W79 replaced with F, or Y; R80 replaced with H, or K; K81 replaced with H, or R; L82 replaced with A, G, I, S, T, M, or V; F83 replaced with W, or Y; S84 replaced with A, G, I, L, T, M, or V; F85 replaced with W, or Y; T86 replaced with A, G, I, L, S, M, or V; K87 replaced with H, or R; Y88 replaced with F, or W; F89 replaced with W, or Y; L90 replaced with A, G, I, S, T, M, or V; K91 replaced with H, or R; I92 replaced with A, G, L, S, T, M, or V; E93 replaced with D; K94 replaced with H, or R; N95 replaced with Q; G96 replaced with A, I, L, S, T, M, or V; K97 replaced with H, or R; V98 replaced with A, G, I, L, S, T, or M; S99 replaced with A, G, I, L, T, M, or V; G100 replaced with A, I, L, S, T, M, or V; T101 replaced with A, G, I, L, S, M, or V; K102 replaced with H, or R; K103 replaced with H, or R; E104 replaced with D; N105 replaced with Q; Y108 replaced with F, or W; S109 replaced with A, G, I, L, T, M, or V; I110 replaced with A, G, L, S, T, M, or V; L111 replaced with A, G, I, S, T, M, or V; E112 replaced with D; I113 replaced with A, G, L, S, T, M, or V; T114 replaced with A, G, I, L, S, M, or V; S115 replaced with A, G, I, L, T, M, or V; V116 replaced with A, G, I, L, S, T, or M; E117 replaced with D; I118 replaced with A, G, L, S, T, M, or V; G119 replaced with A, I, L, S, T, M, or V; V120 replaced with A, G, I, L, S, T, or M; V121 replaced with A, G, I, L, S, T, or M; A122 replaced with G, I, L, S, T, M, or V; V123 replaced with A, G, I, L, S, T, or M; K124 replaced with H, or R; A125 replaced with G, I, L, S, T, M, or V; I126 replaced with A, G, L, S, T, M, or V; N127 replaced with Q; S128 replaced with A, G, I, L, T, M, or V; N129 replaced with Q; Y130 replaced with F, or W; Y131 replaced with F, or W; L132 replaced with A, G, I, S, T, M, or V; A133 replaced with G, I, L, S, T, M, or V; M134 replaced with A, G, I, L, S, T, or V; N135 replaced with Q; K136 replaced with H, or R; K137 replaced with H, or R; G138 replaced with A, I, L, S, T, M, or V; K139 replaced with H, or R; L140 replaced with A, G, I, S, T, M, or V; Y141 replaced with F, or W; G142 replaced with A, I, L, S, T, M, or V; S143 replaced with A, G, I, L, T, M, or V; K144 replaced with H, or R; E145 replaced with D; F146 replaced with W, or Y; N147 replaced with Q; N148 replaced with Q; D149 replaced with E; K151 replaced with H, or R; L152 replaced with A, G, I, S, T, M, or V; K153 replaced with H, or R; E154 replaced with D; R155 replaced with H, or K; I156 replaced with A, G, L, S, T, M, or V; E157 replaced with D; E158 replaced with D; N159 replaced with Q; G160 replaced with A, I, L, S, T, M, or V; Y161 replaced with F, or W; N162 replaced with Q; T163 replaced with A, G, I, L, S, M, or V; Y164 replaced with F, or W; A165 replaced with G, I, L, S, T, M, or V; S166 replaced with A, G, I, L, T, M, or V; F167 replaced with W, or Y; N168 replaced with Q; W169 replaced with F, or Y; Q170 replaced with N; H171 replaced with K, or R; N172 replaced with Q; G173 replaced with A, I, L, S, T, M, or V; R174 replaced with H, or K; Q175 replaced with N; M176 replaced with A, G, I, L, S, T, or V; Y177 replaced with F, or W; V178 replaced with A, G, I, L, S, T, or M; A179 replaced with G, I, L, S, T, M, or V; L180 replaced with A, G, I, S, T, M, or V; N181 replaced with Q; G182 replaced with A, I, L, S, T, M, or V; K183 replaced with H, or R; G184 replaced with A, I, L, S, T, M, or V; A185 replaced with G, I, L, S, T, M, or V; R187 replaced with H, or K; R188 replaced with H, or K; G189 replaced with A, I, L, S, T, M, or V; Q190 replaced with N; K191 replaced with H, or R; T192 replaced with A, G, I, L, S, M, or V; R193 replaced with H, or K; R194 replaced with H, or K; K195 replaced with H, or R; N196 replaced with Q; T197 replaced with A, G, I, L, S, M, or V; S198 replaced with A, G, I, L, T, M, or V; A199 replaced with G, I, L, S, T, M, or V; H200 replaced with K, or R; F201 replaced with W, or Y; L202 replaced with A, G, I, S, T, M, or V; M204 replaced with A, G, I, L, S, T, or V; V205 replaced with A, G, I, L, S, T, or M; V206 replaced with A, G, I, L, S, T, or M; H207 replaced with K, or R; or S208 replaced with A, G, I, L, T, M, or V.

However, also preferred are KGF-2 molecules with nonconservative amino acid substitutions, including: M1 replaced with D, E, H, K, R, N, Q, F, W, Y, P, or C; W2 replaced with D, E, H, K, R, N, Q, A, G, I, L, S, T, M, V, P, or C; K3 replaced with D, E, A, G, I, L, S, T, M, V, N, Q, F, W, Y, P, or C; W4 replaced with D, E, H, K, R, N, Q, A, G, I, L, S, T, M, V, P, or C; I5 replaced with D, E, H, K, R, N, Q, F, W, Y, P, or C; L6 replaced with D, E, H, K, R, N, Q, F, W, Y, P, or C; T7 replaced with D, E, H, K, R, N, Q, F, W, Y, P, or C; H8 replaced with D, E, A, G, I, L, S, T, M, V, N, Q, F, W, Y, P, or C; C9 replaced with D, E, H, K, R, A, G, I, L, S, T, M, V, N, Q, F, W, Y, or P; A10 replaced with D, E, H, K, R, N, Q, F, W, Y, P, or C; S11 replaced with D, E, H, K, R, N, Q, F, W, Y, P, or C; A12 replaced with D, E, H, K, R, N, Q, F, W, Y, P, or C; F13 replaced with D, E, H, K, R, N, Q, A, G, I, L, S, T, M, V, P, or C; P14 replaced with D, E, H, K, R, A, G, I, L, S, T, M, V, N, Q, F, W, Y, or C; H15 replaced with D, E, A, G, I, L, S, T, M, V, N, Q, F, W, Y, P, or C; L16 replaced with D, E, H, K, R, N, Q, F, W, Y, P, or C; P17 replaced with D, E, H, K, R, A, G, I, L, S, T, M, V, N, Q, F, W, Y, or C; G18 replaced with D, E, H, K, R, N, Q, F, W, Y, P, or C; C19 replaced with D, E, H, K, R, A, G, I, L, S, T, M, V, N, Q, F, W, Y, or P; C20 replaced with D, E, H, K, R, A, G, I, L, S, T, M, V, N, Q, F, W, Y, or P; C21 replaced with D, E, H, K, R, A, G, I, L, S, T, M, V, N, Q, F, W, Y, or P; C22 replaced with D, E, H, K, R, A, G, I, L, S, T, M, V, N, Q, F, W, Y, or P; C23 replaced with D, E, H, K, R, A, G, I, L, S, T, M, V, N, Q, F, W, Y, or P; F24 replaced with D, E, H, K, R, N, Q, A, G, I, L, S, T, M, V, P, or C; L25 replaced with D, E, H, K, R, N, Q, F, W, Y, P, or C; L26 replaced with D, E, H, K, R, N, Q, F, W, Y, P, or C; L27 replaced with D, E, H, K, R, N, Q, F, W, Y, P, or C; F28 replaced with D, E, H, K, R, N, Q, A, G, I, L, S, T, M, V, P, or C; L29 replaced with D, E, H, K, R, N, Q, F, W, Y, P, or C; V30 replaced with D, E, H, K, R, N, Q, F, W, Y, P, or C; S31 replaced with D, E, H, K, R, N, Q, F, W, Y, P, or C; S32 replaced with D, E, H, K, R, N, Q, F, W, Y, P, or C; V33 replaced with D, E, H, K, R, N, Q, F, W, Y, P, or C; P34 replaced with D, E, H, K, R, A, G, I, L, S, T, M, V, N, Q, F, W, Y, or C; V35 replaced with D, E, H, K, R, N, Q, F, W, Y, P, or C; T36 replaced with D, E, H, K, R, N, Q, F, W, Y, P, or C; C37 replaced with D, E, H, K, R, A, G, I, L, S, T, M, V, N, Q, F, W, Y, or P; Q38 replaced with D, E, H, K, R, A, G, I, L, S, T, M, V, F, W, Y, P, or C; A39 replaced with D, E, H, K, R, N, Q, F, W, Y, P, or C; L40 replaced with D, E, H, K, R, N, Q, F, W, Y, P, or C; G41 replaced with D, E, H, K, R, N, Q, F, W, Y, P, or C; Q42 replaced with D, E, H, K, R, A, G, I, L, S, T, M, V, F, W, Y, P, or C; D43 replaced with H, K, R, A, G, I, L, S, T, M, V, N, Q, F, W, Y, P, or C; M44 replaced with D, E, H, K, R, N, Q, F, W, Y, P, or C; V45 replaced with D, E, H, K, R, N, Q, F, W, Y, P, or C; S46 replaced with D, E, H, K, R, N, Q, F, W, Y, P, or C; P47 replaced with D, E, H, K, R, A, G, I, L, S, T, M, V, N, Q, F, W, Y, or C; E48 replaced with H, K, R, A, G, I, L, S, T, M, V, N, Q, F, W, Y, P, or C; A49 replaced with D, E, H, K, R, N, Q, F, W, Y, P, or C; T50 replaced with D, E, H, K, R, N, Q, F, W, Y, P, or C; N51 replaced with D, E, H, K, R, A, G, I, L, S, T, M, V, F, W, Y, P, or C; S52 replaced with D, E, H, K, R, N, Q, F, W, Y, P, or C; S53 replaced with D, E, H, K, R, N, Q, F, W, Y, P, or C; S54 replaced with D, E, H, K, R, N, Q, F, W, Y, P, or C; S55 replaced with D, E, H, K, R, N, Q, F, W, Y, P, or C; S56 replaced with D, E, H, K, R, N, Q, F, W, Y, P, or C; F57 replaced with D, E, H, K, R, N, Q, A, G, I, L, S, T, M, V, P, or C; S58 replaced with D, E, H, K, R, N, Q, F, W, Y, P, or C; S59 replaced with D, E, H, K, R, N, Q, F, W, Y, P, or C; P60 replaced with D, E, H, K, R, A, G, I, L, S, T, M, V, N, Q, F, W, Y, or C; S61 replaced with D, E, H, K, R, N, Q, F, W, Y, P, or C; S62 replaced with D, E, H, K, R, N, Q, F, W, Y, P, or C; A63 replaced with D, E, H, K, R, N, Q, F, W, Y, P, or C; G64 replaced with D, E, H, K, R, N, Q, F, W, Y, P, or C; R65 replaced with D, E, A, G, I, L, S, T, M, V, N, Q, F, W, Y, P, or C; H66 replaced with D, E, A, G, I, L, S, T, M, V, N, Q, F, W, Y, P, or C; V67 replaced with D, E, H, K, R, N, Q, F, W, Y, P, or C; R68 replaced with D, E, A, G, I, L, S, T, M, V, N, Q, F, W, Y, P, or C; S69 replaced with D, E, H, K, R, N, Q, F, W, Y, P, or C; Y70 replaced with D, E, H, K, R, N, Q, A, G, I, L, S, T, M, V, P, or C; N71 replaced with D, E, H, K, R, A, G, I, L, S, T, M, V, F, W, Y, P, or C; H72 replaced with D, E, A, G, I, L, S, T, M, V, N, Q, F, W, Y, P, or C; L73 replaced with D, E, H, K, R, N, Q, F, W, Y, P, or C; Q74 replaced with D, E, H, K, R, A, G, I, L, S, T, M, V, F, W, Y, P, or C; G75 replaced with D, E, H, K, R, N, Q, F, W, Y, P, or C; D76 replaced with H, K, R, A, G, I, L, S, T, M, V, N, Q, F, W, Y, P, or C; V77 replaced with D, E, H, K, R, N, Q, F, W, Y, P, or C; R78 replaced with D, E, A, G, I, L, S, T, M, V, N, Q, F, W, Y, P, or C; W79 replaced with D, E, H, K, R, N, Q, A, G, I, L, S, T, M, V, P, or C; R80 replaced with D, E, A, G, I, L, S, T, M, V, N, Q, F, W, Y, P, or C; K81 replaced with D, E, A, G, I, L, S, T, M, V, N, Q, F, W, Y, P, or C; L82 replaced with D, E, H, K, R, N, Q, F, W, Y, P, or C; F83 replaced with D, E, H, K, R, N, Q, A, G, I, L, S, T, M, V, P, or C; S84 replaced with D, E, H, K, R, N, Q, F, W, Y, P, or C; F85 replaced with D, E, H, K, R, N, Q, A, G, I, L, S, T, M, V, P, or C; T86 replaced with D, E, H, K, R, N, Q, F, W, Y, P, or C; K87 replaced with D, E, A, G, I, L, S, T, M, V, N, Q, F, W, Y, P, or C; Y88 replaced with D, E, H, K, R, N, Q, A, G, I, L, S, T, M, V, P, or C; F89 replaced with D, E, H, K, R, N, Q, A, G, I, L, S, T, M, V, P, or C; L90 replaced with D, E, H, K, R, N, Q, F, W, Y, P, or C; K91 replaced with D, E, A, G, I, L, S, T, M, V, N, Q, F, W, Y, P, or C; I92 replaced with D, E, H, K, R, N, Q, F, W, Y, P, or C; E93 replaced with H, K, R, A, G, I, L, S, T, M, V, N, Q, F, W, Y, P, or C; K94 replaced with D, E, A, G, I, L, S, T, M, V, N, Q, F, W, Y, P, or C; N95 replaced with D, E, H, K, R, A, G, I, L, S, T, M, V, F, W, Y, P, or C; G96 replaced with D, E, H, K, R, N, Q, F, W, Y, P, or C; K97 replaced with D, E, A, G, I, L, S, T, M, V, N, Q, F, W, Y, P, or C; V98 replaced with D, E, H, K, R, N, Q, F, W, Y, P, or C; S99 replaced with D, E, H, K, R, N, Q, F, W, Y, P, or C; G100 replaced with D, E, H, K, R, N, Q, F, W, Y, P, or C; T101 replaced with D, E, H, K, R, N, Q, F, W, Y, P, or C; K102 replaced with D, E, A, G, I, L, S, T, M, V, N, Q, F, W, Y, P, or C; K103 replaced with D, E, A, G, I, L, S, T, M, V, N, Q, F, W, Y, P, or C; E104 replaced with H, K, R, A, G, I, L, S, T, M, V, N, Q, F, W, Y, P, or C; N105 replaced with D, E, H, K, R, A, G, I, L, S, T, M, V, F, W, Y, P, or C; C106 replaced with D, E, H, K, R, A, G, I, L, S, T, M, V, N, Q, F, W, Y, or P; P107 replaced with D, E, H, K, R, A, G, I, L, S, T, M, V, N, Q, F, W, Y, or C; Y108 replaced with D, E, H, K, R, N, Q, A, G, I, L, S, T, M, V, P, or C; S109 replaced with D, E, H, K, R, N, Q, F, W, Y, P, or C; I110 replaced with D, E, H, K, R, N, Q, F, W, Y, P, or C; L111 replaced with D, E, H, K, R, N, Q, F, W, Y, P, or C; E112 replaced with H, K, R, A, G, I, L, S, T, M, V, N, Q, F, W, Y, P, or C; I113 replaced with D, E, H, K, R, N, Q, F, W, Y, P, or C; T114 replaced with D, E, H, K, R, N, Q, F, W, Y, P, or C; S115 replaced with D, E, H, K, R, N, Q, F, W, Y, P, or C; V116 replaced with D, E, H, K, R, N, Q, F, W, Y, P, or C; E117 replaced with H, K, R, A, G, I, L, S, T, M, V, N, Q, F, W, Y, P, or C; I118 replaced with D, E, H, K, R, N, Q, F, W, Y, P, or C; G119 replaced with D, E, H, K, R, N, Q, F, W, Y, P, or C; V120 replaced with D, E, H, K, R, N, Q, F, W, Y, P, or C; V121 replaced with D, E, H, K, R, N, Q, F, W, Y, P, or C; A122 replaced with D, E, H, K, R, N, Q, F, W, Y, P, or C; V123 replaced with D, E, H, K, R, N, Q, F, W, Y, P, or C; K124 replaced with D, E, A, G, I, L, S, T, M, V, N, Q, F, W, Y, P, or C; A125 replaced with D, E, H, K, R, N, Q, F, W, Y, P, or C; I126 replaced with D, E, H, K, R, N, Q, F, W, Y, P, or C; N127 replaced with D, E, H, K, R, A, G, I, L, S, T, M, V, F, W, Y, P, or C; S128 replaced with D, E, H, K, R, N, Q, F, W, Y, P, or C; N129 replaced with D, E, H, K, R, A, G, I, L, S, T, M, V, F, W, Y, P, or C; Y130 replaced with D, E, H, K, R, N, Q, A, G, I, L, S, T, M, V, P, or C; Y131 replaced with D, E, H, K, R, N, Q, A, G, I, L, S, T, M, V, P, or C; L132 replaced with D, E, H, K, R, N, Q, F, W, Y, P, or C; A133 replaced with D, E, H, K, R, N, Q, F, W, Y, P, or C; M134 replaced with D, E, H, K, R, N, Q, F, W, Y, P, or C; N135 replaced with D, E, H, K, R, A, G, I, L, S, T, M, V, F, W, Y, P, or C; K136 replaced with D, E, A, G, I, L, S, T, M, V, N, Q, F, W, Y, P, or C; K137 replaced with D, E, A, G, I, L, S, T, M, V, N, Q, F, W, Y, P, or C; G138 replaced with D, E, H, K, R, N, Q, F, W, Y, P, or C; K139 replaced with D, E, A, G, I, L, S, T, M, V, N, Q, F, W, Y, P, or C; L140 replaced with D, E, H, K, R, N, Q, F, W, Y, P, or C; Y141 replaced with D, E, H, K, R, N, Q, A, G, I, L, S, T, M, V, P, or C; G142 replaced with D, E, H, K, R, N, Q, F, W, Y, P, or C; S143 replaced with D, E, H, K, R, N, Q, F, W, Y, P, or C; K144 replaced with D, E, A, G, I, L, S, T, M, V, N, Q, F, W, Y, P, or C; E145 replaced with H, K, R, A, G, I, L, S, T, M, V, N, Q, F, W, Y, P, or C; F146 replaced with D, E, H, K, R, N, Q, A, G, I, L, S, T, M, V, P, or C; N147 replaced with D, E, H, K, R, A, G, I, L, S, T, M, V, F, W, Y, P, or C; N148 replaced with D, E, H, K, R, A, G, I, L, S, T, M, V, F, W, Y, P, or C; D149 replaced with H, K, R, A, G, I, L, S, T, M, V, N, Q, F, W, Y, P, or C; C150 replaced with D, E, H, K, R, A, G, I, L, S, T, M, V, N, Q, F, W, Y, or P; K151 replaced with D, E, A, G, I, L, S, T, M, V, N, Q, F, W, Y, P, or C; L152 replaced with D, E, H, K, R, N, Q, F, W, Y, P, or C; K153 replaced with D, E, A, G, I, L, S, T, M, V, N, Q, F, W, Y, P, or C; E154 replaced with H, K, R, A, G, I, L, S, T, M, V, N, Q, F, W, Y, P, or C; R155 replaced with D, E, A, G, I, L, S, T, M, V, N, Q, F, W, Y, P, or C; I156 replaced with D, E, H, K, R, N, Q, F, W, Y, P, or C; E157 replaced with H, K, R, A, G, I, L, S, T, M, V, N, Q, F, W, Y, P, or C; E158 replaced with H, K, R, A, G, I, L, S, T, M, V, N, Q, F, W, Y, P, or C; N159 replaced with D, E, H, K, R, A, G, I, L, S, T, M, V, F, W, Y, P, or C; G160 replaced with D, E, H, K, R, N, Q, F, W, Y, P, or C; Y161 replaced with D, E, H, K, R, N, Q, A, G, I, L, S, T, M, V, P, or C; N162 replaced with D, E, H, K, R, A, G, I, L, S, T, M, V, F, W, Y, P, or C; T163 replaced with D, E, H, K, R, N, Q, F, W, Y, P, or C; Y164 replaced with D, E, H, K, R, N, Q, A, G, I, L, S, T, M, V, P, or C; A165 replaced with D, E, H, K, R, N, Q, F, W, Y, P, or C; S 166 replaced with D, E, H, K, R, N, Q, F, W, Y, P, or C; F167 replaced with D, E, H, K, R, N, Q, A, G, I, L, S, T, M, V, P, or C; N168 replaced with D, E, H, K, R, A, G, I, L, S, T, M, V, F, W, Y, P, or C; W169 replaced with D, E, H, K, R, N, Q, A, G, I, L, S, T, M, V, P, or C; Q170 replaced with D, E, H, K, R, A, G, I, L, S, T, M, V, F, W, Y, P, or C; H171 replaced with D, E, A, G, I, L, S, T, M, V, N, Q, F, W, Y, P, or C; N172 replaced with D, E, H, K, R, A, G, I, L, S, T, M, V, F, W, Y, P, or C; G173 replaced with D, E, H, K, R, N, Q, F, W, Y, P, or C; R174 replaced with D, E, A, G, I, L, S, T, M, V, N, Q, F, W, Y, P, or C; Q175 replaced with D, E, H, K, R, A, G, I, L, S, T, M, V, F, W, Y, P, or C; M176 replaced with D, E, H, K, R, N, Q, F, W, Y, P, or C; Y177 replaced with D, E, H, K, R, N, Q, A, G, I, L, S, T, M, V, P, or C; V178 replaced with D, E, H, K, R, N, Q, F, W, Y, P, or C; A179 replaced with D, E, H, K, R, N, Q, F, W, Y, P, or C; L180 replaced with D, E, H, K, R, N, Q, F, W, Y, P, or C; N181 replaced with D, E, H, K, R, A, G, I, L, S, T, M, V, F, W, Y, P, or C; G182 replaced with D, E, H, K, R, N, Q, F, W, Y, P, or C; K183 replaced with D, E, A, G, I, L, S, T, M, V, N, Q, F, W, Y, P, or C; G184 replaced with D, E, H, K, R, N, Q, F, W, Y, P, or C; A185 replaced with D, E, H, K, R, N, Q, F, W, Y, P, or C; P186 replaced with D, E, H, K, R, A, G, I, L, S, T, M, V, N, Q, F, W, Y, or C; R187 replaced with D, E, A, G, I, L, S, T, M, V, N, Q, F, W, Y, P, or C; R188 replaced with D, E, A, G, I, L, S, T, M, V, N, Q, F, W, Y, P, or C; G189 replaced with D, E, H, K, R, N, Q, F, W, Y, P, or C; Q190 replaced with D, E, H, K, R, A, G, I, L, S, T, M, V, F, W, Y, P, or C; K191 replaced with D, E, A, G, I, L, S, T, M, V, N, Q, F, W, Y, P, or C; T192 replaced with D, E, H, K, R, N, Q, F, W, Y, P, or C; R193 replaced with D, E, A, G, I, L, S, T, M, V, N, Q, F, W, Y, P, or C; R194 replaced with D, E, A, G, I, L, S, T, M, V, N, Q, F, W, Y, P, or C; K195 replaced with D, E, A, G, I, L, S, T, M, V, N, Q, F, W, Y, P, or C; N196 replaced with D, E, H, K, R, A, G, I, L, S, T, M, V, F, W, Y, P, or C; T197 replaced with D, E, H, K, R, N, Q, F, W, Y, P, or C; S198 replaced with D, E, H, K, R, N, Q, F, W, Y, P, or C; A199 replaced with D, E, H, K, R, N, Q, F, W, Y, P, or C; H200 replaced with D, E, A, G, I, L, S, T, M, V, N, Q, F, W, Y, P, or C; F201 replaced with D, E, H, K, R, N, Q, A, G, I, L, S, T, M, V, P, or C; L202 replaced with D, E, H, K, R, N, Q, F, W, Y, P, or C; P203 replaced with D, E, H, K, R, A, G, I, L, S, T, M, V, N, Q, F, W, Y, or C; M204 replaced with D, E, H, K, R, N, Q, F, W, Y, P, or C; V205 replaced with D, E, H, K, R, N, Q, F, W, Y, P, or C; V206 replaced with D, E, H, K, R, N, Q, F, W, Y, P, or C; H207 replaced with D, E, A, G, I, L, S, T, M, V, N, Q, F, W, Y, P, or C; or S208 replaced with D, E, H, K, R, N, Q, F, W, Y, P, or C.

The substitution mutants can be tested in any of the assays described herein for activity. Particularly preferred are KGF-2 and FGF-12 molecules with conservative substitutions that maintain the activities and properties of the wild type protein; have an enhanced activity or property compared to the wild type protein, while all other activities or properties are maintained; or have more than one enhanced activity or property compared to the wild type protein. In contrast, KGF-2 and FGF-12 molecules with nonconservative substitutions preferably lack an activity or property of the wild type protein, while maintaining all other activities and properties; or lack more than one activity or property of the wild type protein.

For example, activities or properties of KGF-2 that may be altered in KGF-2 molecules with conservative or nonconservative substitutions include, but are not limited to: stimulation of growth of keratinocytes, epithelial cells, hair follicles, hepatocytes, renal cells, breast tissue, bladder cells, prostate cells, pancreatic cells; stimulation of differentiation of muscle cells, nervous tissue, prostate cells, lung cells, hepatocytes, renal cells, breast tissue; promotion of wound healing; angiogenesis stimulation; reduction of inflammation; cytoprotection; heparin binding; ligand binding; stability; solubility; and/or properties which affect purification.

Amino acids in KGF-2 or FGF-12 that are essential for function can be identified by methods well known in the art, such as site-directed mutagenesis or alanine-scanning mutagenesis (Cunningham and Wells, Science 244 :1081-1085 (1989)). The latter procedure introduces single alanine mutations at every residue in the molecule. The resulting mutant molecules are then tested for biological activity such as receptor binding or in vitro and in vivo proliferative activity. Sites that are critical for ligand-receptor binding can also be determined by structural analysis such as crystallization, nuclear magnetic resonance or photoaffinity labelling. (See for example: Smith et al., J. Mol. Biol., 224: 899-904 (1992); and de Vos et al. Science, 255: 306-312 (1992).)

Another aspect of the present invention substitutions of serine for cysteine at amino acid positions 37 and 106 and 150. An uneven number of cysteines means that at least one cysteine residue is available for intermolecular crosslinks or bonds that can cause the protein to adopt an undesirable tertiary structure. Novel KGF-2 proteins that have one or more cysteine replaced by serine or e.g. alanine are generally purified at a higher yield of soluble, correctly folded protein. Although not proven, it is believed that the cysteine residue at position 106 is important for function. This cysteine residue is highly conserved among all other FGF family members.

A further aspect of the present invention is producing fusions of KGF2 or FGF-12 with other proteins or fragments thereof such as fusions or hybrids with other FGF proteins, e.g. KGF (FGF-7), bFGF, aFGF, FGF-5, FGF-6, etc. Such a hybrid has been reported for KGF (FGF-7). In the published PCT application no. 90/08771 a chimeric protein has been produced consisting of the first 40 amino acid residues of KGF and the C-terminal portion of aFGF. The chimera has been reported to target keratinocytes like KGF, but lacked susceptibility to heparin, a characteristic of aFGF but not KGF. Fusions with parts of the constant domain of immunoglobulins (IgG) show often an increased half-life time in vivo. This has been shown, e.g., for chimeric proteins consisting of the first two domains of the human CD4-polypeptide with various domains of the constant regions of the heavy or light chains of mammalian immunoglobulins (European Patent application, Publication No. 394 827, Traunecker et al., Nature 331, 84-86 (1988). Fusion proteins that have a disulfide-linked dimeric structure can also be more efficient in binding monomeric molecules alone (Fountoulakis et al., J. of Biochemistry, 270: 3958-3964 (1995)).

The present invention also relates to vectors which include the isolated DNA molecules of the present invention, host cells which are genetically engineered with the recombinant vectors, and the production of KGF-2 or FGF-12 polypeptides or fragments thereof by recombinant techniques.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the KGF-2 or FGF-12 genes. The culture conditions, such as temperature, pH and the like, are those previously used with the host cell selected for expression, and will be apparent to the ordinarily skilled artisan.

The polynucleotides of the present invention may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequences) (promoter) to direct cDNA synthesis. As representative examples of such promoters, there may be mentioned: LTR or SV40 promoter, the E. coli. lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline, kanamycin or ampicillin resistance in E. coli.

The vector containing the appropriate DNA sequence as hereinabove described, as well as an appropriate promoter or control sequence, may be employed to transform an appropriate host to permit the host to express the protein.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; plant cells, etc. The selection of an appropriate host is deemed to be within the scope of those skilled in the art from the teachings herein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example; Bacterial: pQE70, pQE60, pQE-9 (Qiagen), pBS, pD1O, phagescript, psiX174, pbluescript SK, pbsks, pNH8A, pNH16a, pNH18A, pNH46A (Stratagene); ptrc99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L. et al., Basic Methods in Molecular Biology (1986)). Preferably, the host cell is an ompT-deficient prokaryotic cell. Particularly preferred ompT-deficient prokaryotic cells are gram-negative bacteria, Psudomonas, Klebsiella, E. coli and Salmonella.

The constructs in host cells can be used in a conventional manner to produce the gene product encoded by the recombinant sequence. Alternatively, the polypeptides of the invention can be synthetically produced by conventional peptide synthesizers.

KGF-2 or FGF-12 proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, Second Edition, Cold Spring Harbor, N.Y. (1989), the disclosure of which is hereby incorporated by reference.

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples including the SV40 enhancer on the late side of the replication origin bp 100 to 270, a cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize receptors. For example, EP-A-O 464 533 (Canadian counterpart 2045869) discloses fusion proteins comprising various portions of constant region of immunoglobin molecules together with another human protein or part thereof. In many cases, the Fc part in fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as, shIL5-receptor has been fused with Fc portions for the purpose of high-throughput screening assays to identify antagonists of hIL-5. See, D. Bennett et al., Journal of Molecular Recognition, Vol. 8 52-58 (1995) and K. Johanson et al., The Journal of Biological Chemistry, Vol. 270, No. 16, pp 9459-9471 (1995).

Generally, recombinant expression vectors will include origins of replication and selectable markers permitting transformation of the host cell, e.g., the kanamycin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., USA). These pBR322 “backbone” sections are combined with an appropriate promoter and the structural sequence to be expressed.

Following transformation of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well known to those skilled in the art.

The polypeptide can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the KGF-2 or FGF-12 protein. Finally, high performance liquid chromatography (HPLC) can be employed for final purification steps.

Having generally described the invention, the same will be more readily understood by reference to the following examples, which are provided by way of illustration and are not intended as limiting.

### EXAMPLES

### Example 1

### Keratinocyte Proliferation Assay

Dermal keratinocytes are cells in the epidermis of the skin. The growth and spreading of keratinocytes in the skin is an important process in wound healing. A proliferation assay of keratinocyte is therefore a valuable indicator of protein activities in stimulating keratinocyte growth and consequently, wound healing.

Keratinocytes are, however, difficult to grow in vitro. Few keratinocyte cell lines exist. These cell lines have different cellular and genetic defects. In order to avoid complications of this assay by cellular defects such as loss of key growth factor receptors or dependence of key growth factors for growth, primary dermal keratinocytes are chosen for this assay. These primary keratinocytes are obtained from Clonetics, Inc. (San Diego, Calif.).

AlamarBlue is a viable blue dye that is metabolized by the mitochondria when added to the culture media. The dye then turns red in tissue culture supernatants. The amounts of the red dye may be directly quantitated by reading difference in optical densities between 570 nm and 600 nm. This reading reflects cellular activities and cell number.

Normal primary dermal keratinocytes (CC-0255, NHEK-Neo pooled) are purchased from Clonetics, Inc. These cells are passage 2. Keratinocytes are grown in complete keratinocyte growth media (CC-3001, KGM; Clonetics, Inc.) until they reach 80% confluency. The cells are trypsinized according to the manufacturer's specification. Briefly, cells were washed twice with Hank's balanced salt solution. 2-3 ml of trypsin was added to cells for about 3-5 min at room temperature. Trypsin neutralization solution was added and cells were collected. Cells are spun at 600× g for 5 min at room temperature and plated into new flasks at 3,000 cells per square centimeter using pre-warmed media.

For the proliferation assay, plate 1,000-2,000 keratinocytes per well of the Corning flat bottom 96-well plates in complete media except for the outermost rows. Fill the outer wells with 200 μl of sterile water. This helps to keep temperature and moisture fluctuations of the wells to the minimum. Grow cells overnight at 37° C. with 5% CO₂. Wash cells twice with keratinocyte basal media (CC-3101, KBM, Clonetics, Inc.) and add 100 μl of KBM into each well. Incubate for 24 hours. Dilute growth factors in KBM in serial dilution and add 100 μl to each well. Use KGM as a positive control and KBM as a negative control. Six wells are used for each concentration point. Incubate for two to three days. At the end of incubation, wash cells once with KBM and add 100 μl of KBM with 10% v/v alamarBlue pre-mixed in the media. Incubate for 6 to 16 hours until media color starts to turn red in the KGM positive control. Measure O.D. 570 nm minus O.D. 600 nm by directly placing plates in the plate reader.

### Example 2

### Construction of ompT-Deficient E. coli Host

E. coli strain W3110 was obtained from the American Type Culture Collection (ATCC No. 26325). The gene coding for TonA, a bacteriophage receptor, was deleted through known recombinant techniques. The gene coding for OmpT was then deleted through known recombinant techniques. Bass et al., J. Bact. 178:1154-1161 (1996). The ompT-deficient E. coli strain was deposited as ATCC Deposit No. PTA-3395 on May 21, 2001 at the American Type Culture Collection Patent Depository, 10801 University Boulevard, Manassas, Va. 20110-2209

### Example 3

### Bacterial Expression and Purification of KGF-2

The DNA sequence encoding KGF-2, ATCC Deposit No. 75977, is initially amplified using PCR oligonucleotide primers corresponding to the 5′ and 3′ end sequences of the processed KGF-2 cDNA (including the signal peptide sequence). The 5′ oligonucleotide primer has the sequence:

5′ CCCCACATGTGGAAATGGATACTGACACATTGTGCC 3′ (SEQ ID NO:13) contains an Afl III restriction enzyme site including and followed by 30 nucleotides of KGF-2 coding sequence starting from the presumed initiation codon. The 3′ sequence:

5′ CCCAAGCTTCCACAAACGTTGCCTTCCTCTATGAG 3′ (SEQ ID NO:14) contains complementary sequences to Hind III site and is followed by 26 nucleotides of KGF-2. The restriction enzyme sites are compatible with the restriction enzyme sites on the bacterial expression vector pQE-60 (Qiagen, Inc. Chatsworth, Calif.). pQE-60 encodes antibiotic resistance (Amp^{r}), a bacterial origin of replication (ori), an IPTG-regulatable promoter operator (P/0), a ribosome binding site (RBS), a 6-His tag and restriction enzyme sites. pQE-60 is then digested with NcoI and HindIII. The amplified sequences are ligated into pQE-60 and are inserted in frame. The ligation mixture is then used to transform the ompT E. coli strain described above by the procedure described in Sambrook, J., et al., Molecular Cloning: A Laboratory Manual, Cold Spring Laboratory Press, (1989).

Transformants are identified by their ability to grow on LB plates and ampicillin resistant colonies are selected. Plasmid DNA is isolated and confirmed by restriction analysis. Clones containing the desired constructs are grown overnight (O/N) in liquid culture in LB media supplemented with Amp (100 ug/ml). The O/N culture is used to inoculate a large culture at a ratio of 1:100 to 1:250. The cells are grown to an optical density 600 (O.D.⁶⁰⁰) of between 0.4 and 0.6. IPTG (“Isopropyl-B-D-thiogalacto pyranoside”) is then added to a final concentration of 1 mM. IPTG interacts with the lacI repressor to cause it to dissociate from the operator, forcing the promoter to direct transcription. Cells are grown an extra 3 to 4 hours. Cells are then harvested by centrifugation. The cell pellet is solubilized in the chaotropic agent 6 Molar Guanidine HCl. After clarification, solubilized KGF-2 is purified from this solution by chromatography on a Heparin affinity column under conditions that allow for tight binding of the proteins (Hochuli, E., et al., J. Chromatography 411:177-184 (1984)). KGF-2 (75% pure) is eluted from the column by high salt buffer.

### Example 4

### Construction of E. coli Optimized Full Length KGF-2

In order to increase expression levels of full length KGF-2 in an E. coli expression system, the codons of the amino terminal portion of the gene were optimized to highly used E. coli codons. For the synthesis of the optimized region of KGF-2, a series of six oligonucleotides were synthesized: numbers 1-6 (sequences set forth below). These overlapping oligos were used in a PCR reaction for seven rounds at the following conditions:

| | | |
|---|---|---|
| Denaturation | 95 degrees | 20 seconds |
| Annealing | 58 degrees | 20 seconds |
| Extension | 72 degrees | 60 seconds |

A second PCR reaction was set up using 1 μl of the first PCR reaction with KFG-2 synthetic primer 6 as the 3′ primer and KGF-2 synthetic 5′ BamHI as the 5′ primer using the same conditions as described above for 25 cycles. The product produced by this final reaction was restricted with AvaII and BamHI. The KGF-2 construct of Example 1 was restricted with AvaII and HindIII and the fragment was isolated. These two fragments were cloned into pQE-9 restricted with BamHI and HindIII in a three fragment ligation.

Primers used for constructing the optimized synthetic KGF-2 1/208:

| | | |
|---|---|---|
| KGF-2 Synthetic Primer 1: | ATGTGGAAATGGATACTGACCCACTGCGC | (SEQ ID NO:15) |
| | TTCTGCTTTCCCGCACCTGCCGGGTTGCTGCTGCTGCTGCTTCCTGC | |
| | TGCTGTTTC | |
| KGF-2 Synthetic Primer 2: | CCGGAGAAACCATGTCCTGACCCAGAGCCTG | (SEQ ID NO:16) |
| | GCAGGTAACCGGAACAGAAGAAACCAGGAACAGCAGCAGGAAGC | |
| | AGCAGCA | |
| KGF-2 Synthetic Primer 3: | GGGTCAGGACATGGTYJTCTCCGGAAGCTACC | (SEQ ID NO:17) |
| | AACTCTTCTTCTTCTTCTTTCTCTTCTCCGTCTTCTGCTGGTCGTCACG | |
| KGF-2 Synthetic Primer 4: | GGTGAAAGAGAACAGTTTACGCCAACGA | (SEQ ID NO:18) |
| | ACGTCACCCTGCAGGTGGTTGTAAGAACGAACGTGACGACCAGCA | |
| | GAAGACGG | |
| KGF-2 Synthetic Primer 5: | CGTTGGCGTAAACTGTTCTCTTTCACCAAATA | (SEQ ID NO:19) |
| | CTTCCTGAAAATCGAAAAAAACGGTAAAGTTTCTGGGACCAAA | |
| KGF-2 Synthetic Primer 6: | TTTGGTCCCAGAAACTTTACCGTTTTTTT | (SEQ ID NO:20) |
| | CGATTTTCAG | |
| KGF-2 Synthetic 5′ BamHI: | AAAGGATCCATGTGGAAATGGATACTG | (SEQ ID NO:21) |
| | ACCCACTGC | |

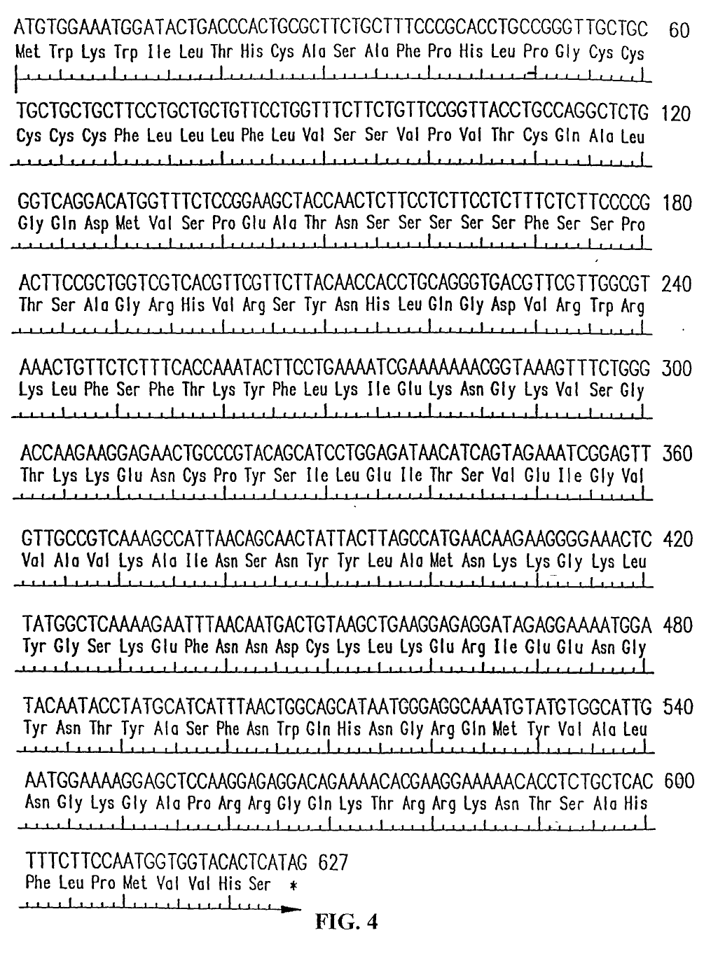

The resulting clone is shown in FIG. 4 (SEQ ID NOs:7 and 8).

### Example 5

### Construction of E. coli Optimized Mature KGF-2

In order to further increase expression levels of the mature form of KGF-2 in an E. coli expression system, the codons of the amino terminal portion of the gene were optimized to highly used E. coli codons. To correspond with the mature form of KGF-1, a truncated form of KGF-2 was constructed starting at threonine 36. E. coli synthetic KGF-2 from Example 12 A was used as a template in a PCR reaction using BspHI 5′ KGF-2 as the 5′ primer (sequence given below) and HindIII 3′ KGF-2 as the 3′ primer (sequence given below). Amplification was performed using standard conditions as given above in Example 4 for 25 cycles. The resulting product was restricted with BspHI and HindIII and cloned into the E. coli expression vector pQE60 digested with NcoI and HindIII.

| | | |
|---|---|---|
| BspHI 5′ KGF-2 Primer: | TTTCATGACTTGTCAAGCTCTGGGTCA | (SEQ ID NO:22) |
| | AGATATGGTTC | |
| HindIII 3′ KGF-2 Primer: | GCCCAAGCTTCCACAAACGTTGCCTCC | (SEQ ID NO:23) |

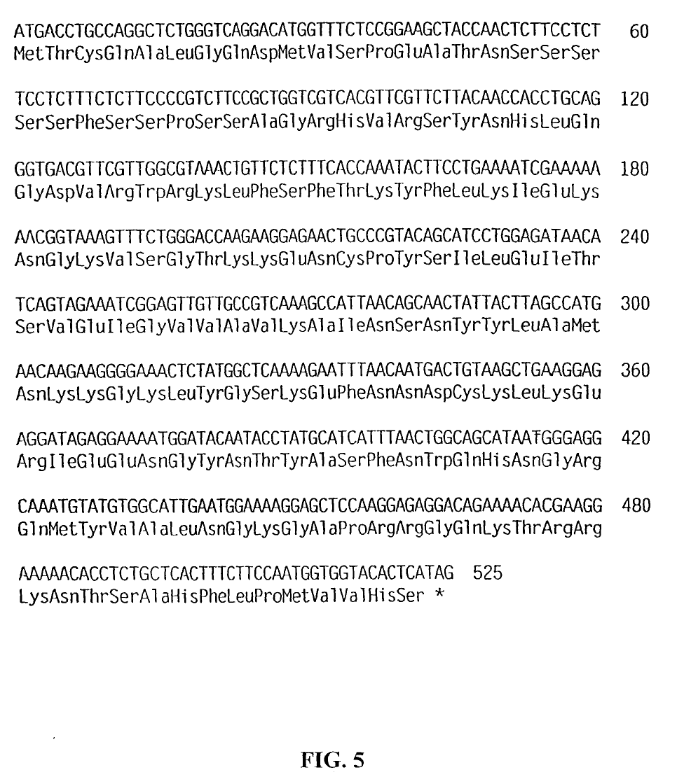

The resulting clone is shown in FIG. 5 (SEQ ID NO:9 and 10).

### Example 6

### Construction of an Alternate E. coli Optimized Mature KGF-2

In order to further increase expression levels of the mature form of KGF-2 in an E. coli expression system, the codons of 53 amino acids at the amino terminal portion of the E. coli optimized gene were changed to alternate highly used E. coli codons. For the synthesis of the optimized region of KGF-2, a series of six oligonucleotides were synthesized: numbers 18062, 18061, 18058, 18064, 18059, and 18063 (sequences set forth below). These overlapping oligos were used in a PCR reaction for seven rounds at the following conditions:

| | | |
|---|---|---|
| Denaturation | 95 degrees | 20 seconds |
| Annealing | 58 degrees | 20 seconds |
| Extension | 72 degrees | 60 seconds |

Following the seven rounds of synthesis, a 5′ primer to this region, 18169 and a 3′ primer to this entire region, 18060, were added to a PCR reaction, containing 1 microliter from the initial reaction of the six oligonucleotides. This product was amplified for 30 rounds using the following conditions:

| | | |
|---|---|---|
| Denaturation | 95 degrees | 20 seconds |
| Annealing | 55 degrees | 20 seconds |
| Extension | 72 degrees | 60 seconds |

A second PCR reaction was set up to amplify the 3′ region of the gene using primers 18066 and 18065 under the same conditions as described above for 25 rounds. The resulting products were separated on an agarose gel. Gel slices containing the product were diluted in 10 mM Tris, 1 mM EDTA, pH 7.5 One microliter each from each of diluted gel slices were used in an additional PCR reaction using primer 18169 as the 5′ primer, and primer 18065 as the 3′ primer. The product was amplified for 25 cycles using the same conditions as above. The product produced by this final reaction was and restricted with EcoR1 and HindIII, and cloned into pQE60, which was also cut with EcoR1 and HindIII (pQE6 now).

Sequences of the 5′ Synthetic Primers:

| | | |
|---|---|---|
| 18169 KGF2 5′ EcoRI/RBS: | TCAGTGAATTCATTAAAGAGGAGAAAT | (SEQ ID NO:24) |
| | TAATCATGACTTGCCAGG | |
| 18062 KGF2 synth new R1 sense: | TCATGACTTGCCAGGCACTGGGTCAAG | (SEQ ID NO:25) |
| | ACATGGTTTCCCCGGAAGCTA | |
| 18061 KGF2 synth R2 sense: | GCTTCAGCAGCCCATCTAGCGCAGGT | (SEQ ID NO:26) |
| | CGTCACGTTCGCTCTTACAACC | |
| 18058 KGF2 Synth R3 sense: | GTTCGTTGGCGCAAACTGTTCAGCT | (SEQ ID NO:27) |
| | TTACCAAGTACTTCCTGAAAATC | |
| 18066 KGF2 20 bp Ava II sense: | TCGAAAAAAACGGTAAAGTTT | (SEQ ID NO:28) |
| | CTGGGAC | |
| 18064 KGF2 synth F1 antisense: | GATGGGCTGCTGAAGCTAGAGCTGGAGC | (SEQ ID NO:29) |
| | TGTTTGGTAGCTTTCCGGGGAA | |
| 18059 KGF2 Synth F2 antisense: | AACAGTTTGCGCCAACGAACAT | (SEQ ID NO:30) |
| | CACCCTGTAAGTGGTTGTAAGAG | |
| 18063 KGF2 Synth F3 antisense: | TTCTTGGTCCCAGAAACTTTACCGTTTTT | (SEQ ID NO:31) |
| | TTCGATTTTCAGGAAGTA | |
| 18060 KGF2 Ava II antisense: | TTCTTGGTCCCAGAAACTTTACCG | (SEQ ID NO:32) |
| 18065 KGF2 HindIII 3′ Stop: | AGATCAGGCTTCTATTATTATGAGTGTACC | (SEQ ID NO:33) |
| | ACCATTGGAAGAAAG | |

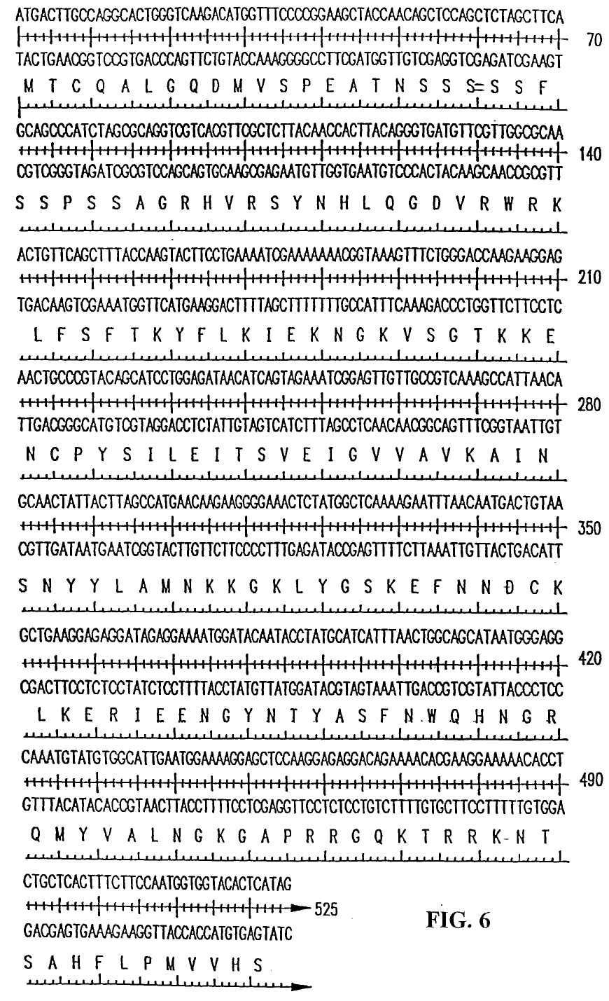

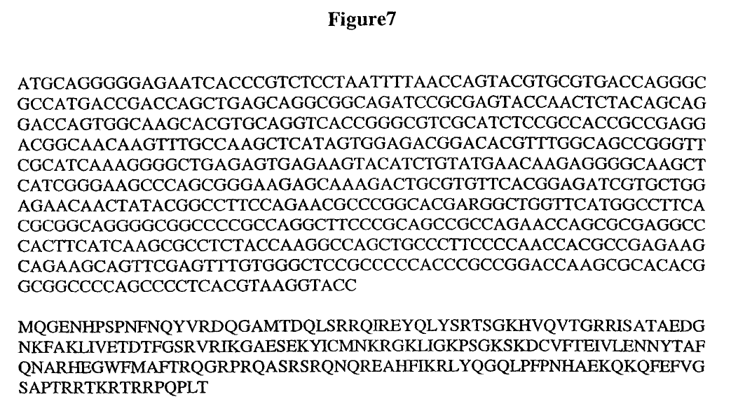

The sequence of the synthetic KGF-2 gene and it corresponding amino acid is shown in FIG. 6 (SEQ ID NO:11 and 12).

### Example 7

### Construction of KGF-2 Deletion Mutants

Deletion mutants were constructed from the 5′ terminus and 3′ terminus of KGF-2 gene using the optimized KGF-2 construct from Example 4 as a template. The deletions were selected based on regions of the gene that might negatively affect expression in E. coli. For the 5′ deletion the primers listed below were used as the 5′ primer. These primers contain the indicated restriction site and an ATG to code for the initiator methionine. The KGF-2 208 amino acid 3′ HindIII primer was used for the 3′ primer. PCR amplification for 25 rounds was performed using standard conditions as set forth in Example 4. The products for the KGF-2 36aa/208aa deletion mutant were restricted BspHI for the 5′ site and HindIII for the 3′ site and cloned into the pQE60 which has been digested with BspHI and HindIII. All other products were restricted with NcoI for the 5′ restriction enzyme and HindIII for the 3′ site, and cloned into the pQE60 which had been digested with NcoI and HindIII. For KGF-2 (FGF-12),36aa/153aa and 128aa 3′ HindIII was used as the 3′ primer with FGF-12 36aa/208aa as the 5′ primer. For FGF-12 62aa/153aa, 128aa 3′ HindIII was used as the 3′ primer with FGF-12 62aa/208aa as the 5′ primer. The nomenclature of the resulting clones indicates the first and last amino acid of the polypeptide that results from the deletion. For example, KGF-2 36aa/153aa indicates that the first amino acid of the deletion mutant is amino acid 36 and the last amino acid is amino acid 153 of KGF-2. Further, as indicated in FIGS. 25-33, each mutant has N-terminal Met added thereto.

Sequences of the Deletion Primers:

| | |
|---|---|
| KGF-2 36aa/208aa: 5′ BsphI | |
| GGACCCTCATGACCTGCCAGGC | (SEQ ID NO:34) |
| TCTGGGTCAGGAC | |
| KGF-2 63aa/208aa: 5′ NcoI | |
| GGACAGCCATGGCTGGTCGTCACGTTCG | (SEQ ID NO:35) |

KGF-2 3′ HindIII: (Used for all above deletion

| | |
|---|---|
| clones) | |
| CTGCCCAAGCTTA | (SEQ ID NO:36) |
| TTATGAGTGTACCACCATTGGAAG | |
| KGF-2 36aa/153aa: | |
| 5′ Bsphl (as above) | |
| 3′HindIII | |
| CTGCCCAAGCTTATTACTTCAGCTTACAGTCATTGT | (SEQ ID NO:37) |

KGF-2 63aa/153aa: 5′NcoI and 3′HindIII, as above

### Example 8

### Construction of Cysteine Mutants of KGF-2

Construction of C-37 mutation primers 5457 5′ BsphI and 5258 173aa 3′ HindIII were used to amplify the KGF-2 template from Example 4. Primer 5457 5′ BsphI changes cysteine 37 to a serine. Amplification was done using the standard conditions outlined above in Example 4 for 25 cycles. The resulting product was restricted with BspHI and HindIII and cloned into E. coli expression vector pQE60, digested with BspHI and HindIII.

For mutation of Cysteine 106 to serine, two PCR reactions were set up for oligonucleotide site directed mutagenesis of this cysteine. In one reaction, 5453 BsphI was used as the 5′ primer, and 5455 was used as the 3′ primer in the reaction. In a second reaction, 5456 was used as the 5′ primer, and 5258 HindIII was used as the 3′ primer. The reactions were amplified for 25 rounds under standard conditions as set forth in Example 4. One microliter from each of these PCR reactions was used as template in a subsequent reaction using, as a 5′ primer, 5453 BspHI, and as a 3′ primer, 5258 HindIII. Amplification for 25 rounds was performed using standard conditions as set forth in Example 4. The resulting product was restricted with BspHI and HindIII and cloned into the E. coli expression vector pQE60, which was restricted with NcoI and HindIII.

Two PCR reactions were required to make the C-37/C-106 mutant. Primers 5457 Bsph1 and 5455 were used to create the 5′ region of the mutant containing cysteine 37 to serine substitution, and primer 5456 and 5258 HindIII were used to create the 3′ region of the mutant containing cysteine 106 to serine substitution. In the second reaction, the 5457 BsphI primer was used as the 5′ primer and the 5258 HindIII primer was used as the 3′ primer to create the C-37/C-106 mutant using 1 μl from each of the initial reactions together as the template. This PCR product was restricted with BsphI and HindIII, and cloned into pQE60 that had been restricted with NcoI and HindIII.

Sequences of the Cysteine Mutant Primers:

| | |
|---|---|
| 5457 BspHI: | (SEQ ID NO:38) |

GGACCCTCATGACCTCTCAGGCTCTGGGT

| | |
|---|---|
| 5456: | (SEQ ID NO:39) |

AAGGAGAACTCTCCGTACAGC

| | |
|---|---|
| 5455: | (SEQ ID NO:40) |

GCTGTACGGTCTGTTCTCCTT

| | |
|---|---|
| 5453 BspHI: | (SEQ ID NO:41) |

GGACCCTCATGACCTGCCAGGCTCTGGGTCAGGAC

| | |
|---|---|
| 5258 HindIII: | (SEQ ID NO:42) |

CTGCCCAAGCTTATTATGAGTGTACCACCATTGGAAG

### Example 9

### Construction of E. coli Optimized Truncated KGF-2

In order to increase expression levels of a truncated KGF-2 in an E. coli expression system, the codons of the gene were optimized to highly used E. coli codons.

For example, the following construct, termed pHE4:KGF-2.A63-S208, was made.

(SEQ ID NO:43)

5′ CATATGGCTGGTCGTCACGTTCGTTCTTACAACCACCTGCAGGGT
GACGTTCGTTGGCGTAAACTGTTCTCTTTCACCAAATACTTCCTGAA
AATCGAAAAAAACGGTAAAGTTTCTGGGACCAAGAAGGAGAACTG
CCCGTACAGCATCCTGGAGATAACATCAGTAGAAATCGGAGTTGTT
GCCGTCAAAGCCATAACAGCAACTATTTACTTAGCCATGAACAAGA
AGGGGAAACTCTATGGCTCAAAAGAATTTAACAATGACTGTAAGCT
GAAGGAGAGGATAGAGGAAAATGGATACAATACCTATGCATCATT
TAACTGGCAGCATAATGGGAGGCAAATGTATGTGGCATTGAATGG
AAAAGGAGCTCCAAGGAGAGGACAGAAAACACGAAGGAAAAACA
CCTCTGCTCACTTTCTTCCAATGGTGGTACACTCATAATAAGGTACC
3′

A plasmid comprising a cDNA having the nucleotide sequence of SEQ ID NO:43 was deposited as ATCC Deposit No. PTA-2183 on Jul. 3, 2000, at the American Type Culture Collection, Patent Depository, 10801 University Boulevard, Manassas, Va. 20110-2209.

Another construct, termed pHE4:KGF-2.A63-S208 cod.opt, was constructed using the following primers:

| | |
|---|---|
| sense | (SEQ ID NO:44) |

5′ GACTACATATGGCTGGTCGTCACGTTCGTTCTTACAACC
ACCTGCA GG 3′

| | |
|---|---|
| antisense | (SEQ ID NO:45) |

5′ CTAGTCTCTAGATTATTATGAGTGTACAACCATCG
GCAGGAAGTGAG 3′

The nucleotide sequence of the pHE4:KGF-2.A63-S208 cod.opt is as follows:

(SEQ ID NO:46)

5′ ATGGCTGGTCGTCACGTTCGTTCTTACAACCACCTGCAGGGTG
ACGTTCGTTGGCGTAAACTGTTCTCTTTCACCAAATACTTCCTGAAA
ATCGAAAAGAACGGTAAAGTTTCTGGTACCAAGAAAGAAAACTGC
CCGTACTCTATCCTGGAAATCACCTCCGTTGAAATCGGTGTTGTAG
CCGTFfAAAGCCATCAACTCCAACTATTACCTGGCCATGAACAAAAA
GGGTAAACTGTACGGCTCTAAAGAATTCAACAACGACTGCAAACT
GAAAGAACGTATCGAAGAGAACGGTTACAACACCTACGCATCCTT
CAACTGGCAGCACAACGGTCGTCAGATGTACGTTGCACTGAACGGT
AAAGGCGCTCCGCGTCGCGGTCAGAAAACCCGTCGCAAAAACACC
TCTGCTCACTTCCTGCCGATGGTTGTACACTCATAATAA 3′

A plasmid comprising a cDNA having the nucleotide sequence of SEQ ID NO:46 was deposited as ATCC Deposit No. PTA-2184 on Jul. 3, 2000, at the American Type Culture Collection, Patent Depository, 10801 University Boulevard, Manassas, Va. 20110-2209.

Both constructs described in this example are useful in the production of KGF-2 polypeptides. Nucleotides 4 to 444 of SEQ ID NO:7 and nucleotides 1 to 441 of SEQ ID NO:10 encode amino acids 63 to 208 of SEQ ID NO:4, plus an N-terminal methionine.

### Example 10

### Expression of the KGF-2 Δ28 in an ompT-Deficient E.coli Host Abolishes the N-Terminal Cleavage of the KGF-2 Protein

A polynucleotide construct encoding KGF-2Δ28 was made (pHE4:KGF-2.A63-S208), as shown in Example 9. The pHE4:KGF-2.A63-S208, plasmid was transfected into ompT-deficient E. coli cells, as made in Example 2. Kan^{R} colonies were selected at 37° C. Clones containing the desired constructs are grown overnight (O/N) in liquid culture in LB media supplemented with Kan. The O/N culture is used to inoculate a large culture at a ratio of 1:100 to 1:250.

The cells were grown to an optical density 600 (O.D.⁶⁰⁰) of between 0.4 and 0.6. IPTG was then added to a final concentration of 1 mM. Cells were grown an extra 3 to 4 hours. Cells were then harvested by centrifugation. The cell pellet was solubilized in the chaotropic agent 6 Molar Guanidine HCl. After clarification, solubilized KGF-2 is purified from this solution by chromatography on a Heparin affinity column under conditions that allow for tight binding of the proteins (Hochuli, E., et al., J. Chromatography 411:177-184 (1984)). KGF-2 (75% pure) is eluted from the column by high salt buffer.

SDS-PAGE and Western blotting analysis was performed to confirm that the KGF-2 polypeptide was not degraded. N-terminal sequencing was performed to confirm the digestion site.

### Example 11

### Expression of FGF-12 in an ompT Deficient E. coli Host Abolishes the C-Terminal Cleaving of the FGF-12 Protein

A pQE vector containing an FGF-12 polynucleotide(SEQ ID NO:6) was made. The pQE-FGF-12 plasmid was transfected into ompT deficient E. coli cells, as made in Example 2. Amp^{R} colonies were selected at 37° C. Clones containing the desired constructs were grown overnight (O/N) in liquid culture in LB media supplemented with Amp. The O/N culture was used to inoculate a large culture at a ratio of 1:100 to 1:250.

The cells were grown to an optical density 600 (O.D.⁶⁰⁰) of between 0.4 and 0.6. IPTG was then added to a final concentration of 1 mM. Cells were grown an extra 3 to 4 hours. Cells were harvested by centrifugation. The cell pellet was solubilized in the chaotropic agent 6 Molar Guanidine HCl. After clarification, solubilized FGF-12 was purified from this solution by chromatography on a Heparin affinity column under conditions that allow for tight binding of the proteins (Hochuli, E., et al., J. Chromatography 411:177-184 (1984)). fGF-12 (75% pure) is eluted from the column by high salt buffer.

SDS-PAGE and Western blotting analysis was performed to confirm that the FGF-12 polypeptide was not degraded. N-terminal sequencing was performed to confirm the digestion site.

### Example 12

### Expression of FGF-13 in an ompT Deficient E. coli Host Abolishes the Cleaving of the FGF-13 Protein

A vector containing an FGF-13 polynucleotide(SEQ ID NO:47) is made using conventional techniques, and transfected into ompT deficient E. coli cells, as made in Example 2. Antibiotic-resistant colonies are selected at 37° C. Clones containing the desired constructs are grown overnight (O/N) in liquid culture in LB media supplemented with antibiotic. The O/N culture is used to inoculate a large culture at a ratio of 1:100 to 1:250.

The cells are grown to an optical density 600 (O.D.⁶⁰⁰) of between 0.4 and 0.6. IPTG is then added to a final concentration of 1 mM. Cells are grown an extra 3 to 4 hours. Cells are harvested by centrifugation. The cell pellet is solubilized in the chaotropic agent 6 Molar Guanidine HCl. After clarification, solubilized FGF-13 is purified from this solution by chromatography on a Heparin affinity column under conditions that allow for tight binding of the proteins (Hochuli, E., et al., J. Chromatography 411:177-184 (1984)). FGF-13 is eluted from the column by high salt buffer.

SDS-PAGE and Western blotting analysis iss performed to confirm that the FGF-13 polypeptide was not degraded. N-terminal sequencing is performed to confirm the digestion site.

It will be clear that the invention may be practiced otherwise than as particularly described in the foregoing description and examples.

Numerous modifications and variations of the present invention are possible in light of the above teachings and, therefore, within the scope of the appended claims, the invention may be practiced otherwise than as particularly described.

The entire disclosure of all publications (including patents, patent applications, journal articles, laboratory manuals, books, or other documents) cited herein are hereby incorporated by reference.

What is claimed is:

1. An ompT deficient prokaryotic host cell comprising a nucleic acid comprising a polynucleotide encoding amino acids 68 to 208 of SEQ ID NO:2.

2. The host cell of claim 1, wherein said polynucleotide encodes amino acids 63 to 208 of SEQ ID NO:2.

3. The host cell of claim 2, wherein said polynucleotide encodes amino acids 37 to 208 of SEQ ID NO:2.

4. The host cell of claim 3, wherein said polynucleotide encodes amino acids 1 to 208 of SEQ ID NO:2.

5. The host cell of claim 1, which is E. coli.

6. A method for producing a KGF-2 polypeptide comprising:
(a) culturing the host cell of claim 1 under conditions sufficient to produce said polypeptide; and
(b) recovering said polypeptide.

7. An ompT deficient prokaryotic host cell comprising a nucleic acid comprising a polypeptide, wherein, except for 1 to 50 amino acids substitutions, deletions, or additions, said polypeptide is selected from the group consisting of:
(a) amino acids 1 to 208 of SEQ ID NO:2;
(b) amino acids 37 to 208 of SEQ ID NO:2;
(c) amino acids 63 to 208 of SEQ ID NO:2; and
(d) amino acids 68 to 208 of SEQ ID NO:2,
and wherein said polynucleotide encodes a polypeptide which stimulates proliferation of keratinocytes.

8. The host cell of claim 7, which is E. coli.

9. A method for producing a KGF-2 polypeptide comprising:
(a) culturing the host cell of claim 7 under conditions sufficient to produce said polypeptide; and
(b) recovering said polypeptide.

10. An ompT deficient prokaryotic host cell comprising a polynucleotide encoding amino acids 1 to 243 of FGF-12.

11. The host cell of claim 10 which is E. coli.

12. The host cell of claim 10, wherein said polynucleotide is SEQ ID NO:5.

13. A method for producing an FGF-12 polypeptide comprising:
(c) culturing the host cell of claim 10 under conditions sufficient to produce said FGF-12 polypeptide; and
(d) recovering said polypeptide.
